(19) United States
(12) Patent Application Publication
(10) Pub. No.: US 2012/0142763 A1
(43) Pub. Date: Jun. 7, 2012

(54) NUCLEIC ACID DELIVERY COMPOSITIONS AND METHODS OF USE THEREOF

(75) Inventors: Steven F. Dowdy, La Jolla, CA (US); Bryan R. Meade, San Diego, CA (US); Khirud Gogoi, Assam (IN)

(73) Assignee: THE REGENTS OF THE UNIVERSITY OF CALIFORNIA, Oakland, CA (US)

(21) Appl. No.: 13/375,451

(22) PCT Filed: Jun. 1, 2010
(86) PCT No.: PCT/US10/36905

§ 371 Date: Jan. 10, 2012

Related U.S. Application Data
(60) Provisional application No. 61/182,832, filed on Jun. 1, 2009.

(51) Int. Cl. A61K 31/7125; C07H 19/067; C12N 5/071; C07H 21/02; C07K 14/00; C07H 1/00; C07H 19/06; C07H 21/00

(52) U.S. Cl. 514/44 R; 536/26.8; 536/26.7; 536/26.5; 536/23.1; 536/24.5; 530/358; 536/25.34; 435/375

(57) ABSTRACT

This disclosure relates to nucleic acid constructs modified to have a reduced net anionic charge. The constructs comprise phosphotriester and/or phosphothioate protecting groups. The disclosure also provides methods of making and using such constructs.

### CROSS REFERENCE TO RELATED APPLICATIONS

This application claims priority to U.S. Provisional Application Ser. No. 61/182,832 filed Jun. 1, 2009, the disclosure of which is incorporated herein by reference.

### TECHNICAL FIELD

This invention relates to compositions and methods for transducing cells.

### BACKGROUND

Nucleic acid delivery to cells both in vitro and in vivo has been performed using various recombinant viral vectors, lipid delivery systems and electroporation. Such techniques have sought to treat various diseases and disorders by knocking-out gene expression, providing genetic constructs for gene therapy or to study various biological systems.

Polyanionic oligomers such as oligonucleotides do not readily diffuse across cell membranes. In order to overcome this problem for cultured cells, cationic lipids are combined with anionic oligonucleotides to assist uptake. Unfortunately, this complex is generally toxic to cells, which means that both the exposure time and concentration of cationic lipid must be carefully controlled to insure transfection of viable cells.

The discovery of RNA interference (RNAi) as a cellular mechanism that selectively degrades mRNAs allows for both the targeted manipulation of cellular phenotypes in cell culture and the potential for development of directed therapeutics (Behlke, Mol. Ther. 13, 644-670, 2006; Xie et al., Drug Discov. Today 11, 67-73, 2006). However, due to their size and negative (anionic) charged nature, siRNAs are macromolecules with no ability to enter cells. Indeed, siRNAs are 25× in excess of Lipinski's “Rule of 5s” for cellular delivery of membrane diffusible molecules that generally limits size to less than 500 Da. Consequently, in the absence of a delivery vehicle or transfection agent, naked siRNAs do not enter cells, even at millimolar concentrations (Barquinero et al., Gene Ther. 11 Suppl 1, S3-9, 2004). Significant attention has been focused on the use of cationic lipids that both condense the siRNA and punch holes in the cellular membrane to solve the siRNA delivery problem. Although widely used, transfection reagents fail to achieve efficient delivery into many cell types, especially primary cells and hematopoietic cell lineages (T and B cells, macrophage). Moreover, lipofection reagents often result in varying degrees of cytotoxicity ranging from mild in tumor cells to high in primary cells.

### SUMMARY

The disclosure provides methods and compositions for delivering masked oligonucleotides or polynucleotides into living cells. The disclosure provides transiently protected oligonucleotides or polynucleotides comprising an anionic charge neutralizing moiety/group. In one embodiment the charge neutralizing moiety comprises a basic/cationic charge. In another embodiment, the moiety comprises a primary, secondary or tertiary amine along a trimester protecting group of the disclosure. These compounds can enter the cytosol of living cells by endocytic or macropinocytic mechanisms. In one embodiment, the phosphotriester protecting/neutralizing group when exposed to the intracellular environment is designed to be removed by enzymatic activity or by passive intracellular methods (e.g., changes in pH) to provide oligonucleotides or polynucleotides capable of eliciting an RNAi response. Accordingly, the disclosure provides oligonucleotide prodrugs useful as therapeutics, diagnostics and as tools for research.

The disclosure provides an RNAi inducing single, soluble RiboNucleic Basic (siRNB) molecule. In some embodiments, the siRNB molecule is conjugated to a Peptide Transduction Domain (PTD) cellular delivery peptide (PTD-siRNB) that is <2×10⁴ Da. RNB phosphoramidite building blocks were engineered that contain biologically reversible, amino isobutyl S-acyl thio ethyl basic phosphotriesters that are specifically removed by cytoplasmic thioesterases resulting in reversion to wild type phosphodiesters. Self-delivering PTD-siRNBs induced rapid RNAi responses in the entire population of primary and transformed cells in culture, and induced RNAi responses in the nasal and upper respiratory passages in mouse models in vivo. PTD-siRNBs represent a novel, single soluble molecule approach to induction of RNAi responses.

The disclosure provides a basic (positive) charge in the form of a primary amine group (pKa >9.0) that makes the RNB soluble in water and is not chased out of solution by a PTD delivery peptide.

The disclosure provides modified nucleotides for use in charge neutralized oligonucleotides. The nucleotide comprises an amino alkyl S-acyl thio alkyl (“charge neutralizing moiety” or “N-SATE”) conjugated to the phosphate group of the nucleotide. The neutralizing group assists in transport of an oligonucleotide comprising the modified nucleotide across a cell membrane. Once taken up by a cell the neutralizing group is removed by, for example, an endogenous or exogenous thio esterase.

In one embodiment a building block for addition of an N-SATE to an oligonucleotide (e.g., and RNA oligonucleotide comprises a charge neutralizing moiety having the general structure:

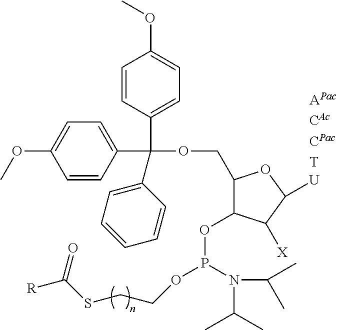

wherein R is an amino group or a 1 to 7 atom alkyl, substituted alkyl, alkoxy, substituted alkoxy, cycloalkyl, substituted cycloalkyl, alkenyl, substituted alkenyl, alkynyl, substituted alkynyl, aryl, substituted aryl, heterocyclic, or substituted heterocyclic terminating in an amino group. The foregoing structure can be added during RNA synthesis reactions to generate an siRNB molecule.

In one embodiment, the charge neutralizing moiety comprises and N-SATE having the general structure:

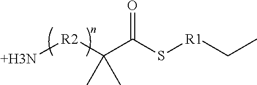

wherein R1 may or may not be present, when R1 is present, R1 is selected from the group consisting of alkyl, substituted alkyl, alkoxy, substituted alkoxy, cycloalkyl, substituted cycloalkyl, alkenyl, substituted alkenyl, alkynyl, substituted alkynyl, aryl, substituted aryl, heterocyclic, or substituted heterocyclic;
wherein R2 may or may not be present, when R2 is present, R2 is selected from the group consisting of a 1 to 7 atom alkyl, substituted alkyl, alkoxy, substituted alkoxy, cycloalkyl, substituted cycloalkyl, alkenyl, substituted alkenyl, alkynyl, substituted alkynyl, aryl, substituted aryl, heterocyclic, or substituted heterocyclic. In one embodiment, the charge neutralizing moiety is selected from the group consisting of:

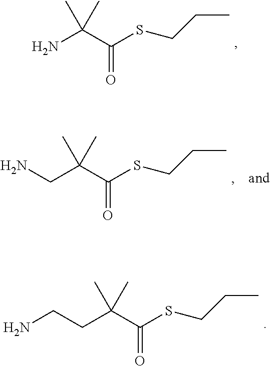

The charge neutralizing moiety can be conjugated to the phosphate group of any of the nucleic acid bases (i.e., A, G, T, U, C). For example, the disclosure provides a nucleotide having the general structure:

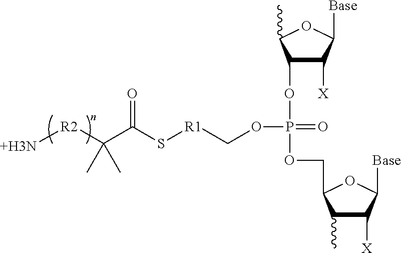

wherein R1 may or may not be present, when R1 is present, R1 is selected from the group consisting of alkyl, substituted alkyl, alkoxy, substituted alkoxy, cycloalkyl, substituted cycloalkyl, alkenyl, substituted alkenyl, alkynyl, substituted alkynyl, aryl, substituted aryl, heterocyclic, or substituted heterocyclic;
wherein R2 may or may not be present, when R2 is present, R2 is selected from the group consisting of a 1 to 7 atom alkyl, substituted alkyl, alkoxy, substituted alkoxy, cycloalkyl, substituted cycloalkyl, alkenyl, substituted alkenyl, alkynyl, substituted alkynyl, aryl, substituted aryl, heterocyclic, or substituted heterocyclic. In one embodiment, the charge neutralizing moiety is selected from the group consisting of:

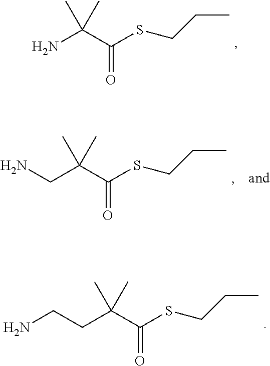

The disclosure further provides a nucleic acid construct comprising an oligonucleotide or polynucleotide comprising a charge neutralizing moiety (e.g., a phosphodiester and/or phosphothioate protecting group) that reduces the net anionic charge of the oligonucleotide or polynucleotide backbone. In one embodiment, the oligonucleotide or polynucleotide comprise an siRNA molecule. In yet another embodiment, the oligonucleotide comprises a plurality modified nucleotides having a charge neutralizing moiety. In yet another embodiment, the oligonucleotide or polynucleotide comprises a plurality of adjacent nucleotides having a charge neutralizing moiety. In yet a another embodiment, the oligonucleotide or polynucleotide comprises a plurality of nucleotides having charge neutralizing moieties separated from one another by 1 or more (e.g., 2, 3, 4, 5, 6, 7, 8, 9 or more nucleotide bases). In yet another embodiment, the oligonucleotide or polynucleotide comprising a charge neutralizing moiety is conjugated or operably linked to a transduction domain comprising a membrane transport function operably linked to the oligonucleotide or polynucleotide domain.

The disclosure also provides pharmaceutical composition comprising the nucleic acid constructs described herein.

The disclosure describes a method comprising linking one or more protein transduction domain to a nucleic acid construct. In one aspect, the one or more protein transduction domains comprise 2-5 protein transduction domains.

The disclosure also provides a method of generating a nucleic acid construct comprising: substantially purifying a protein transduction domain; synthesizing an oligonucleotide; charge neutralizing the anionic charge on the oligonucleotide with a charge neutralizing group; and linking the oligonucleotide to one or more protein transduction domains.

Also provided are methods of transfecting a cell, comprising contacting the cell with a nucleic acid construct of the disclosure. The contacting can be in vivo or in vitro.

The disclosure also provides a method of treating a disease or disorder comprising administering a nucleic acid construct of the disclosure to a subject, wherein the oligonucleotide or polynucleotide comprises a therapeutic or diagnostic molecule.

### BRIEF DESCRIPTION OF THE DRAWINGS

FIG. 1 depicts charge-neutralized oligonucleotides uptake by cells and enzymatic deprotection.

FIG. 2 depicts N-SATE phosphotriester deprotection.

FIG. 3 shows an embodiment a phosphoramidite incorporation into an RNA oligonucleotide.

FIG. 4A-B shows a nucleoside phosphoramidite synthesis route of the disclosure.

FIG. 5 shows Fmoc-N1-SATE purification and characterization.

FIG. 6 shows Fmoc-N1-SATE U phosphoramidite purification and characterization.

FIG. 7 shows Fmoc-N1-SATE C phosphoramidite purification and characterization.

FIG. 8 shows Fmoc-N1-SATE A phosphoramidite purification and characterization.

FIG. 9 shows Fmoc-N2-SATE purification and characterization.

FIG. 10 shows Fmoc-N2-SATE U phosphoramidite purification and characterization.

FIG. 11 shows synthesis of Fmoc-Ala-SATE phosphoramidite.

FIG. 12 shows Fmoc-Ala-SATE purification and characterization.

FIG. 13 shows Fmoc-Ala-SATE U Phosphoramidite purification and characterization.

FIG. 14 shows a test sequence comprising SEQ ID NO:21 having N1-U-SATE nucleotides and purification and characterization.

FIG. 15 shows a test sequence comprising SEQ ID NO:22 having N1-U-SATE nucleotides and purification and characterization.

FIG. 16 shows an RNAi against GFP comprising SEQ ID NO:23 having N1-U-SATE nucleotides and dose response curves showing inhibition of expression of GFP at 24 hours.

FIG. 17 shows an RNAi against GFP comprising SEQ ID NO:23 having N1-U-SATE nucleotides and dose response curves showing inhibition of expression of GFP at 48 hours.

FIG. 18 shows GFP RNAi inhibition in cells.

FIG. 19 shows HPLC purification of AS-10 N1-SATE (SEQ ID NO:24).

FIG. 20 shows HPLC purification results for 3S-9 N1-SATE (SEQ ID NO:25).

FIG. 21 shows purification of S and AS N1-SATE oligonucleotides in a denaturing gel analysis.

FIG. 22 shows purified S-9N1-SATE (SEQ ID NO:25) and AS-10-N1-SATE (SEQ ID NO:24) forms dsRNB.

FIG. 23 shows the 24 hour GFP expression measurements in the presence of various siRNA inhibitors and concentrations (SEQ ID NOs: 24 and 25).

FIG. 24 shows the 48 hour GFP expression measurements in the presence of various siRNA inhibitors and concentrations (SEQ ID NOs: 24 and 25).

FIG. 25 shows the 48 hour GFP expression measurements in the presence of various siRNA inhibitors and concentrations (SEQ ID NOs: 24 and 25).

FIG. 26 shows expression inhibition using charge protected siRNA preparations.

FIG. 27 shows synthesis tester N2-SATE phosphotriester oligonucleotides (SEQ ID NO:21).

FIG. 28 shows the purification of SEQ ID NO:25 comprising 9 charge neutralizing moieties.

FIG. 29 shows the purification of SEQ ID NO:25 comprising 15 charge neutralizing moieties.

FIG. 30 shows the deprotection reaction of SEQ ID NO:25.

FIG. 31 shows the RNAi response using a 21 mer charge-protected oligonucleotide (SEQ ID NO:23) against GFP.

FIG. 32 shows that a charge-protected oligonucleotide (SEQ ID NO:23) using charge-protecting groups of the disclosure are soluble in salt solution.

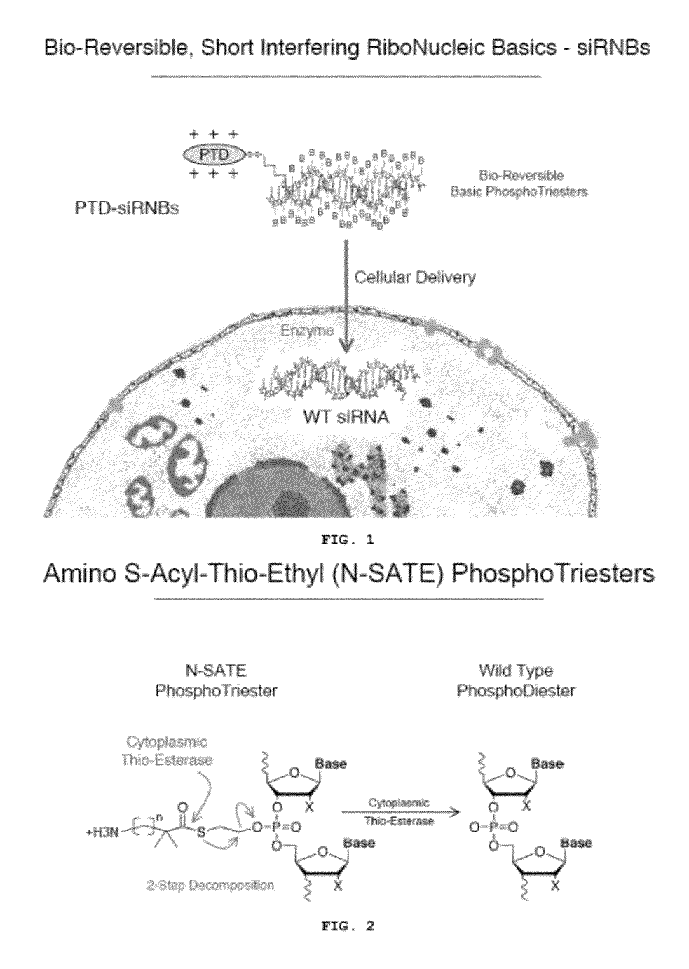

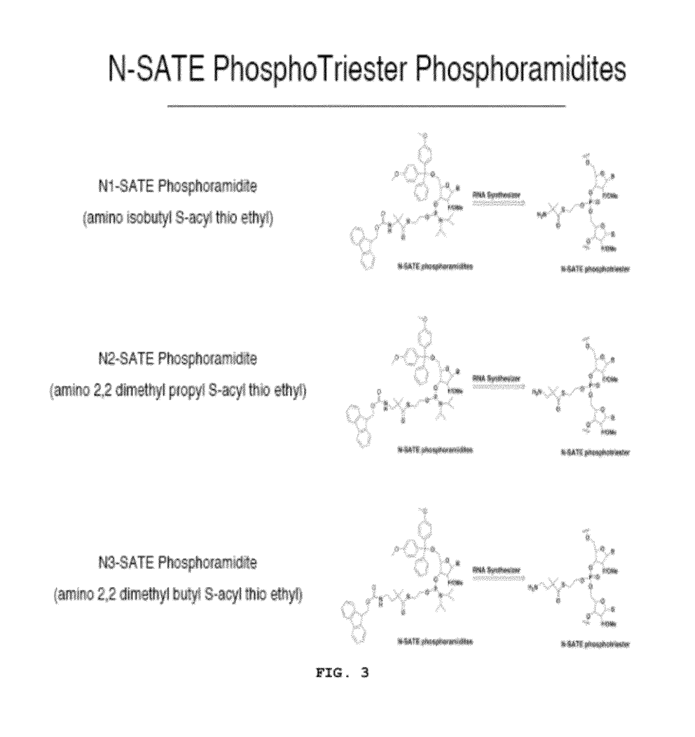

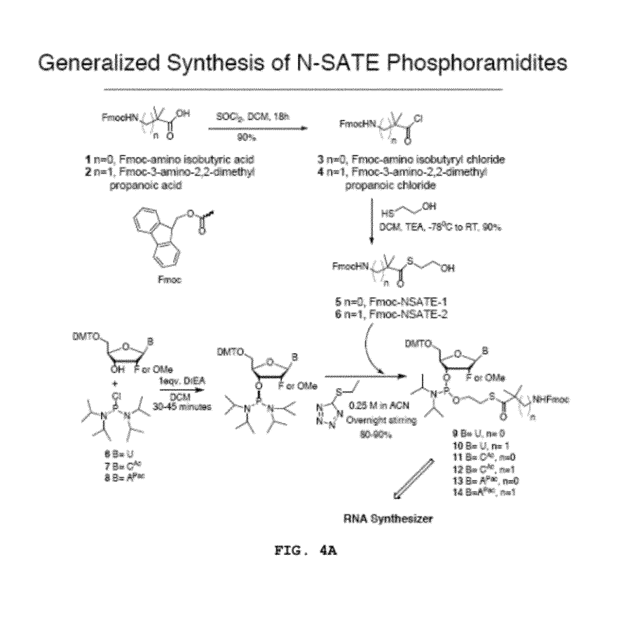

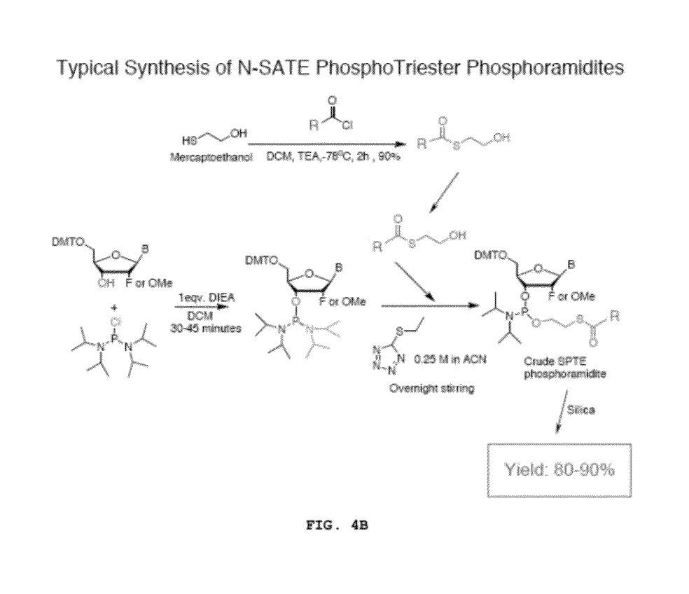

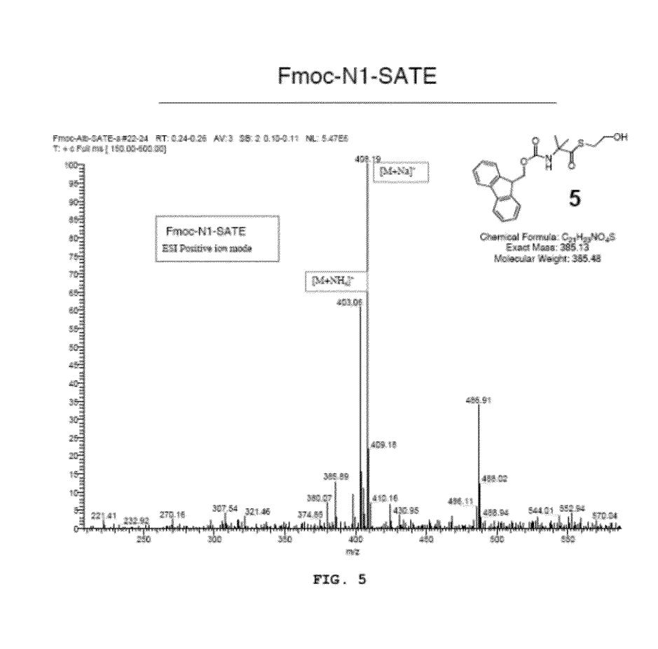

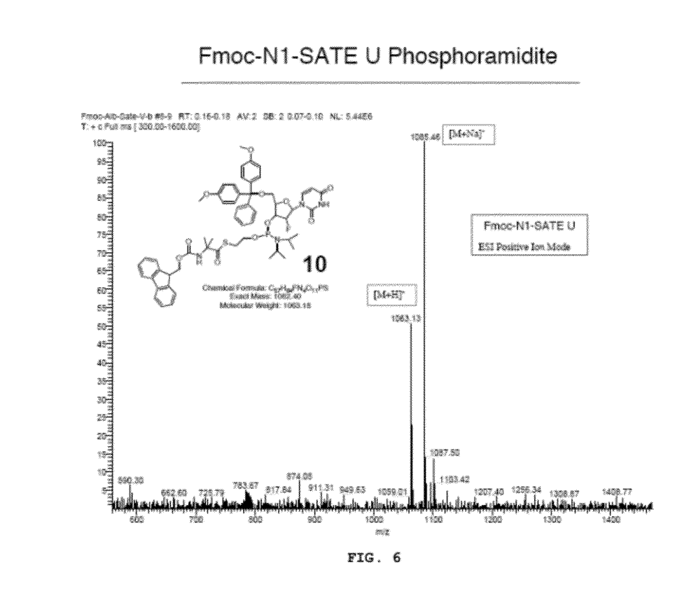

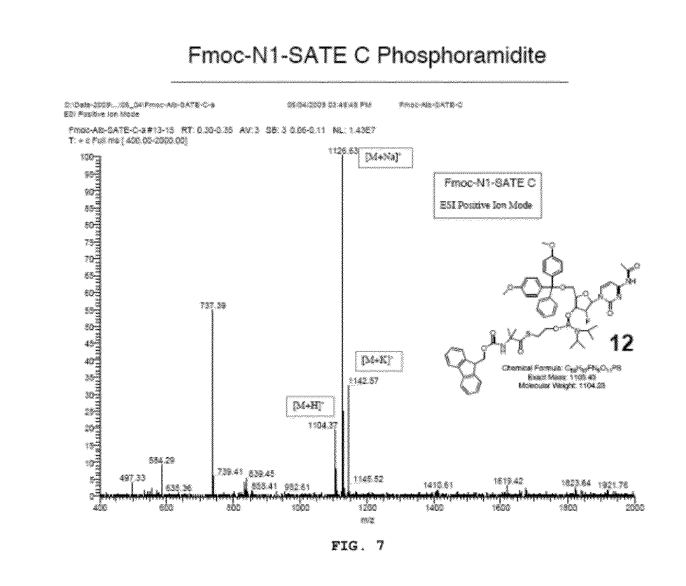

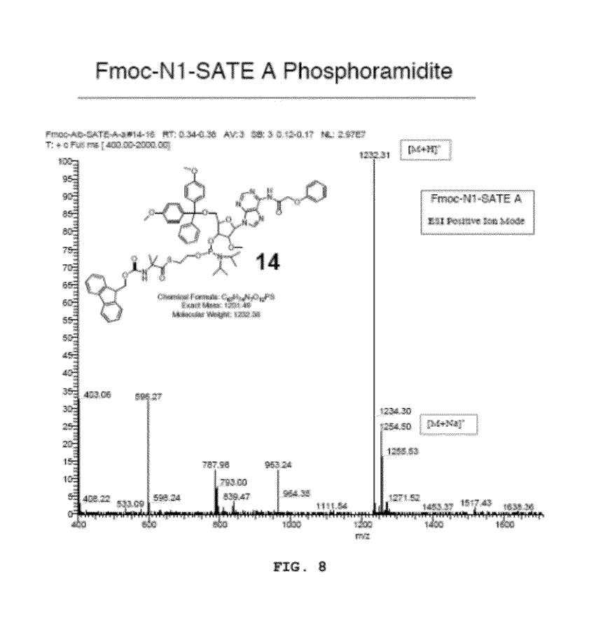

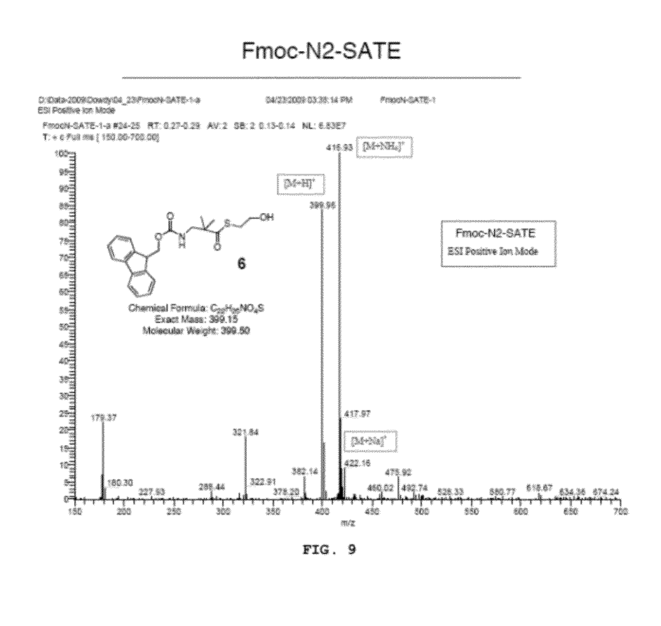

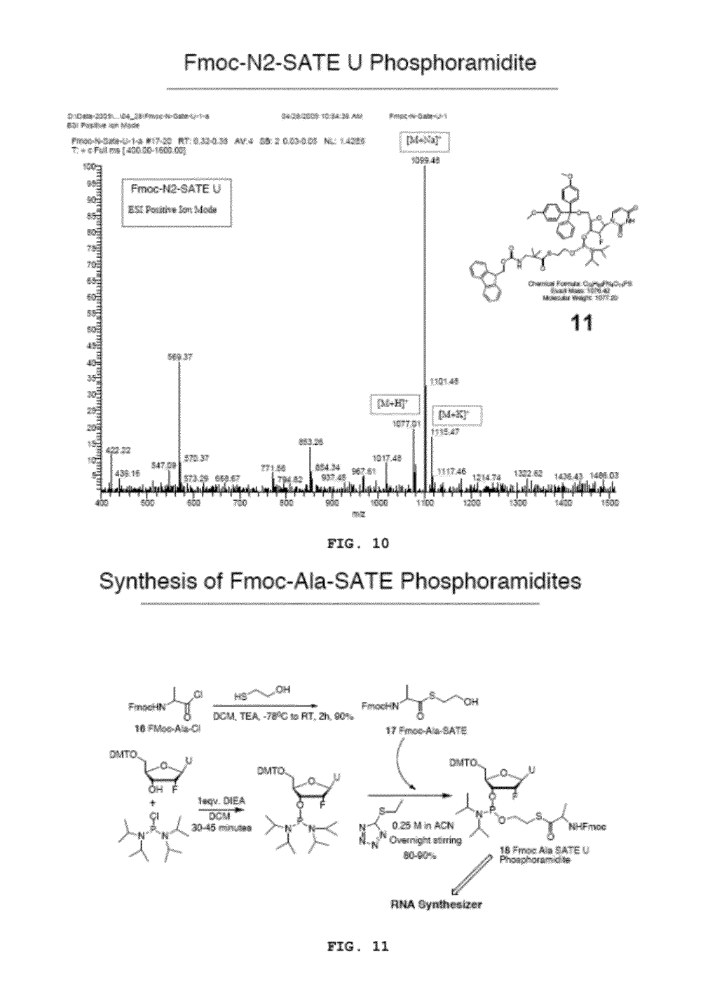

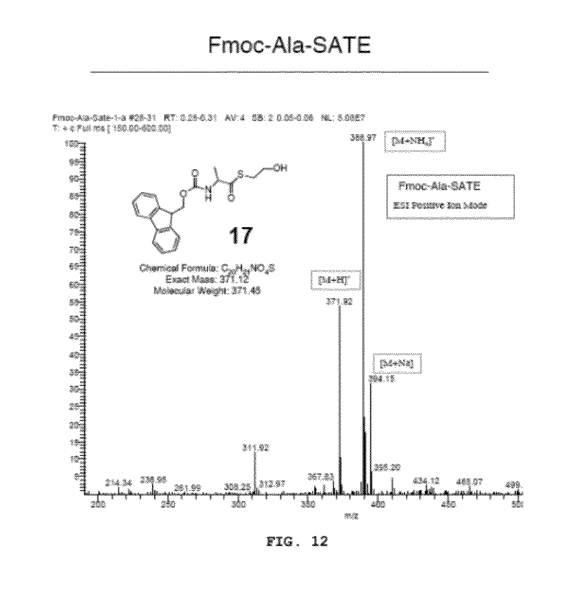

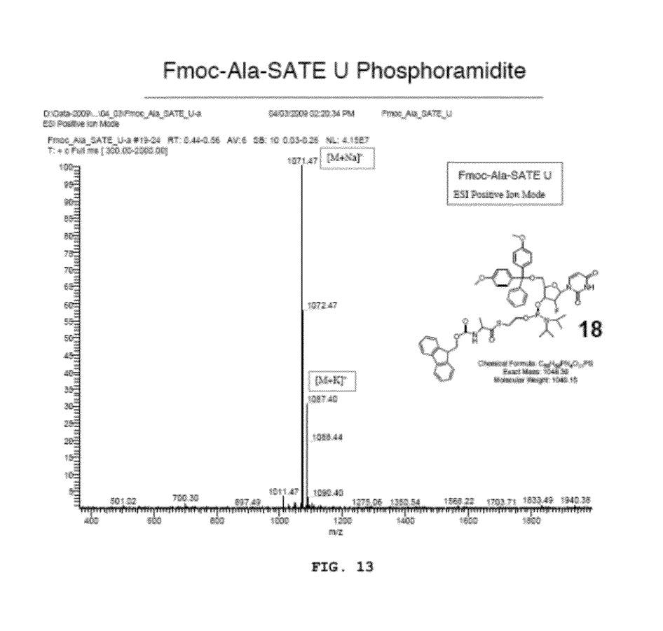

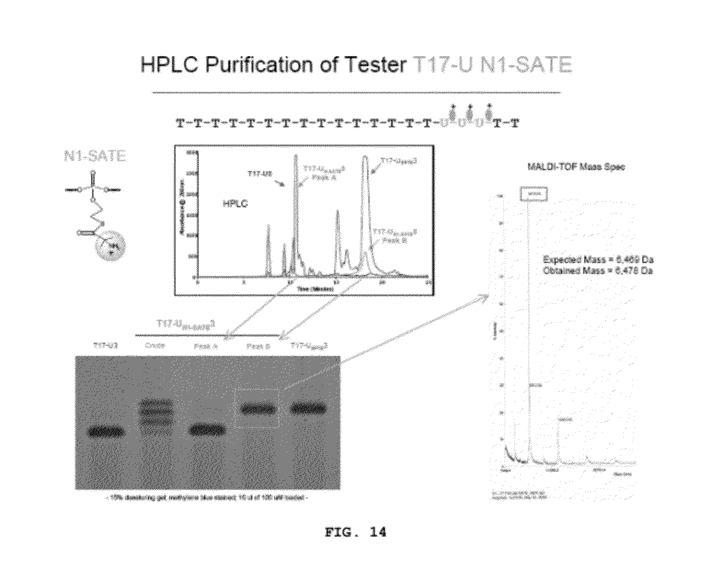

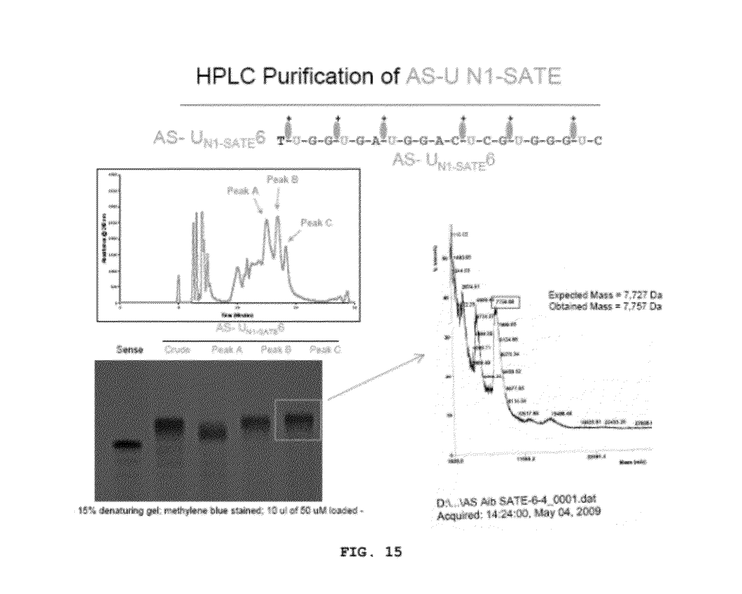

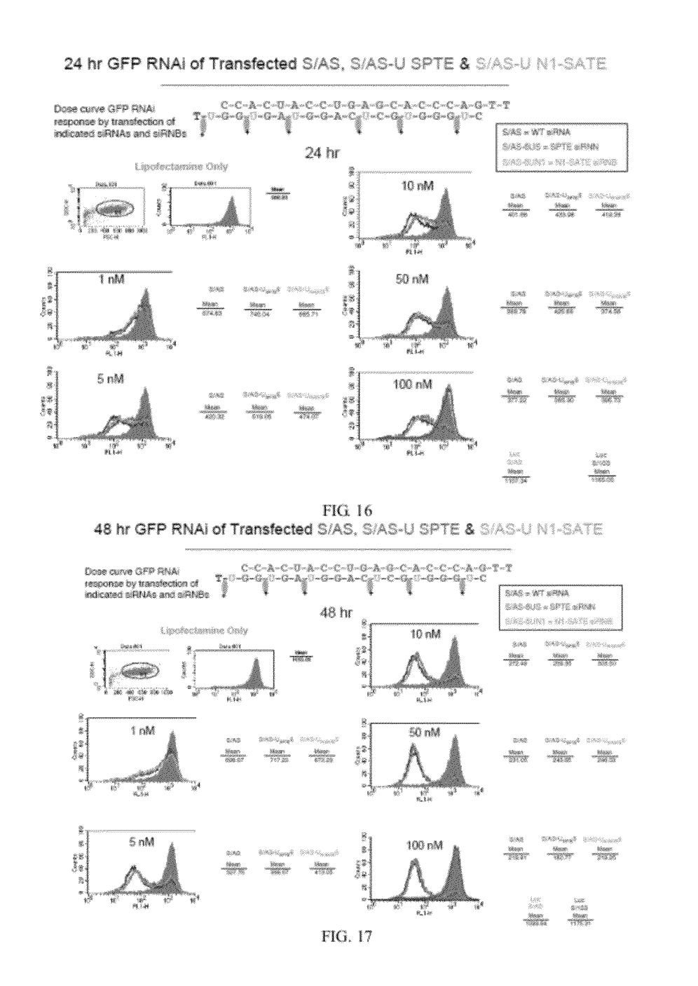

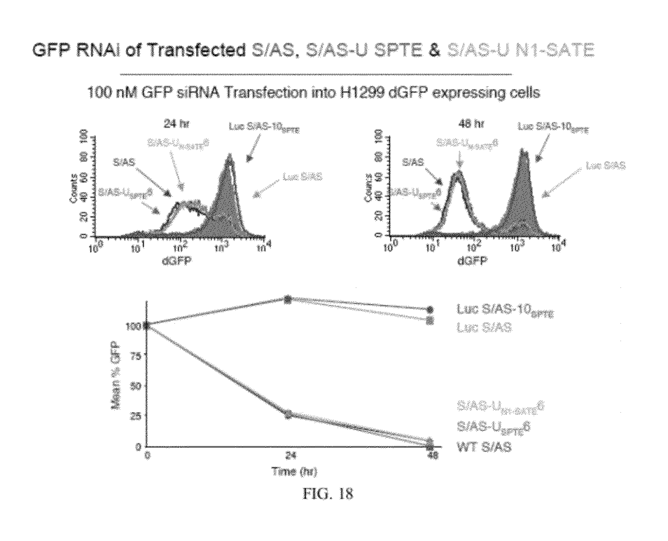

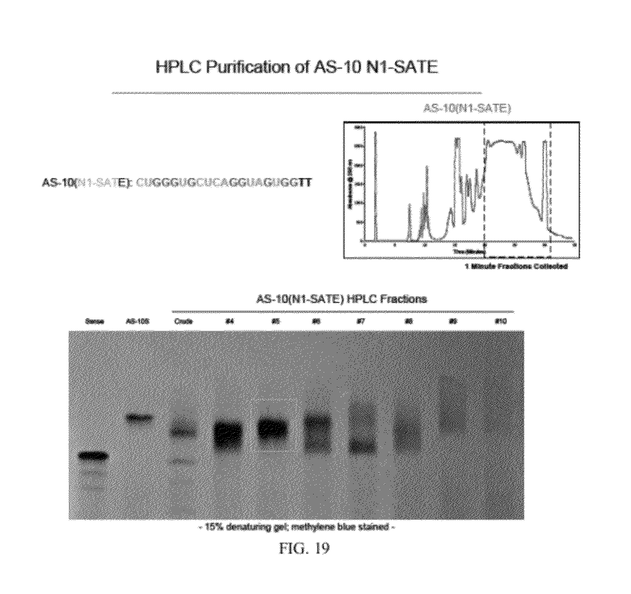

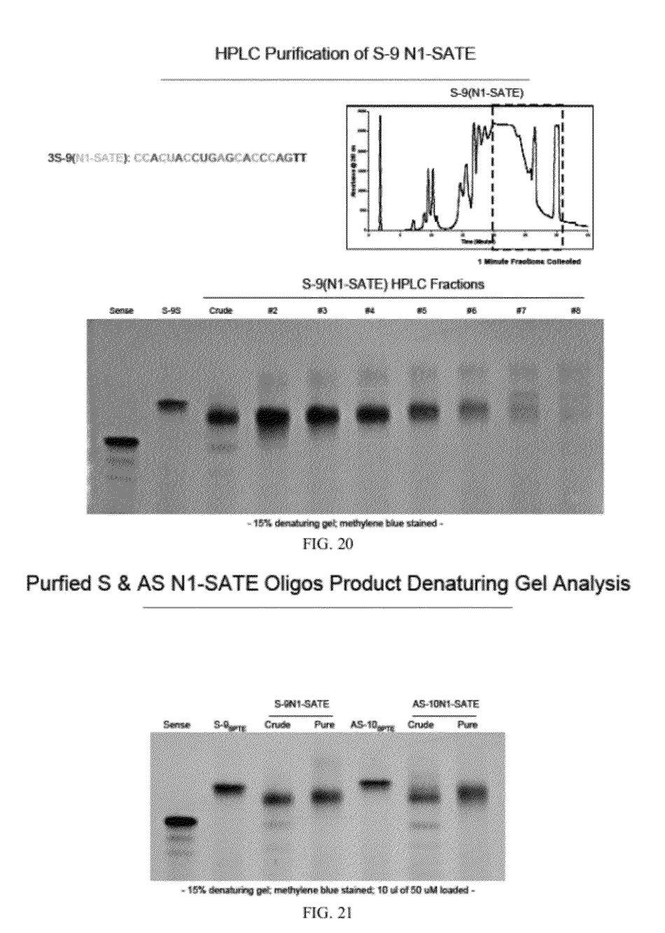

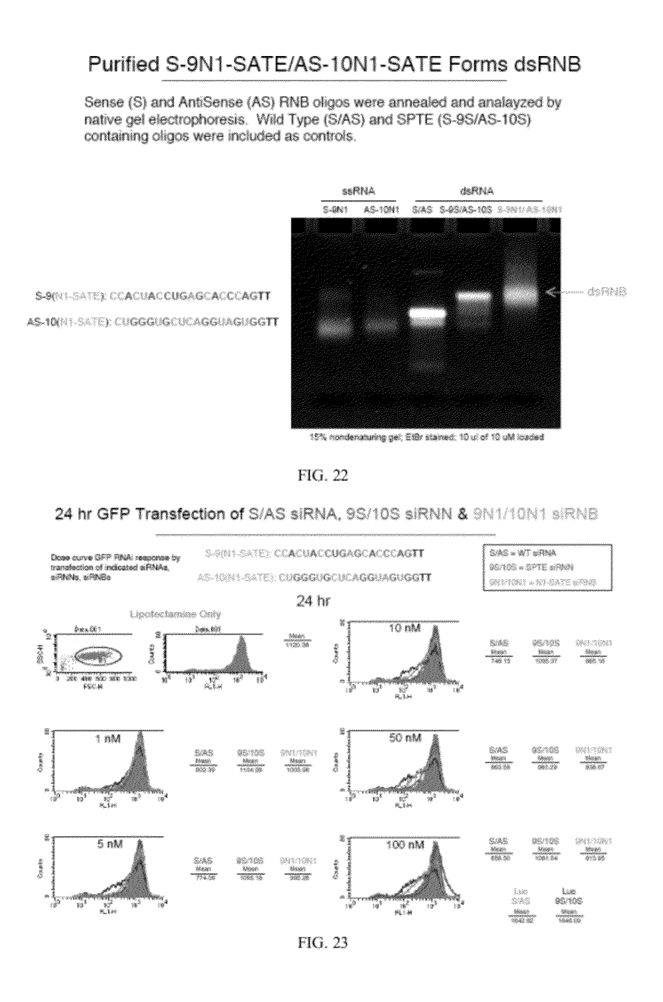

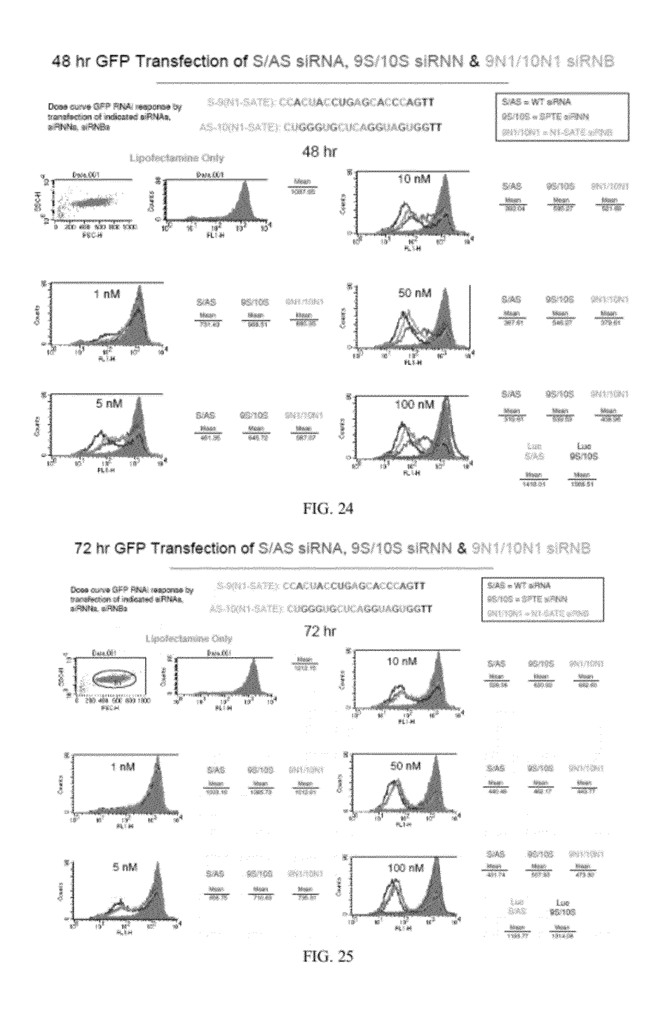

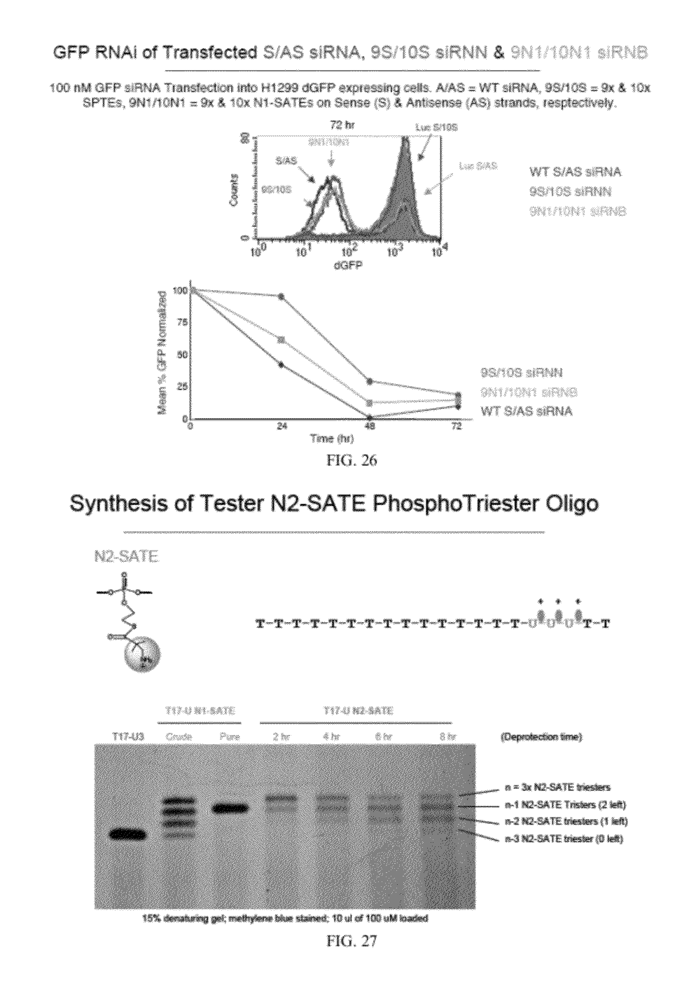

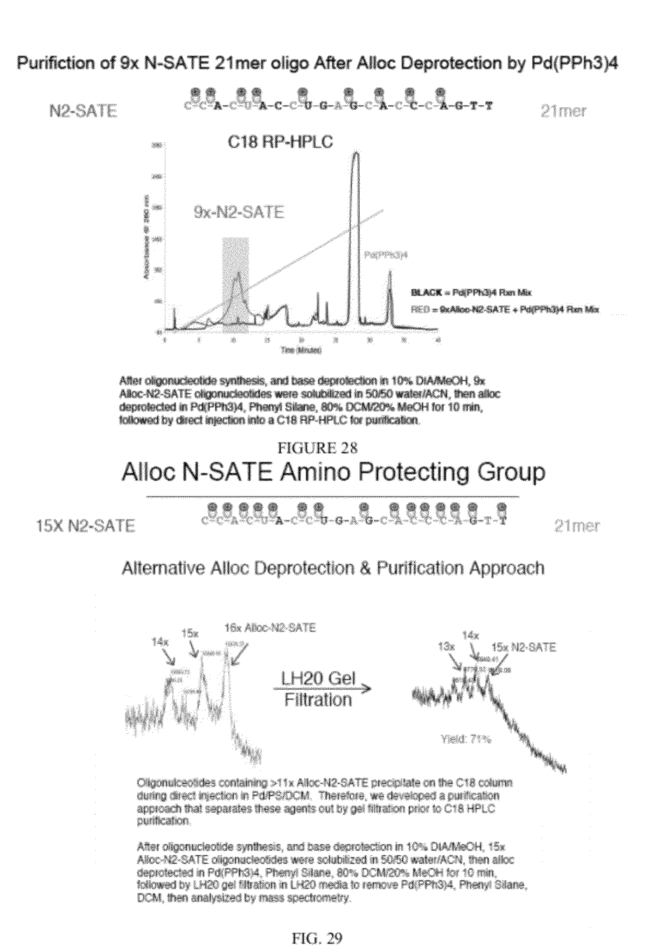

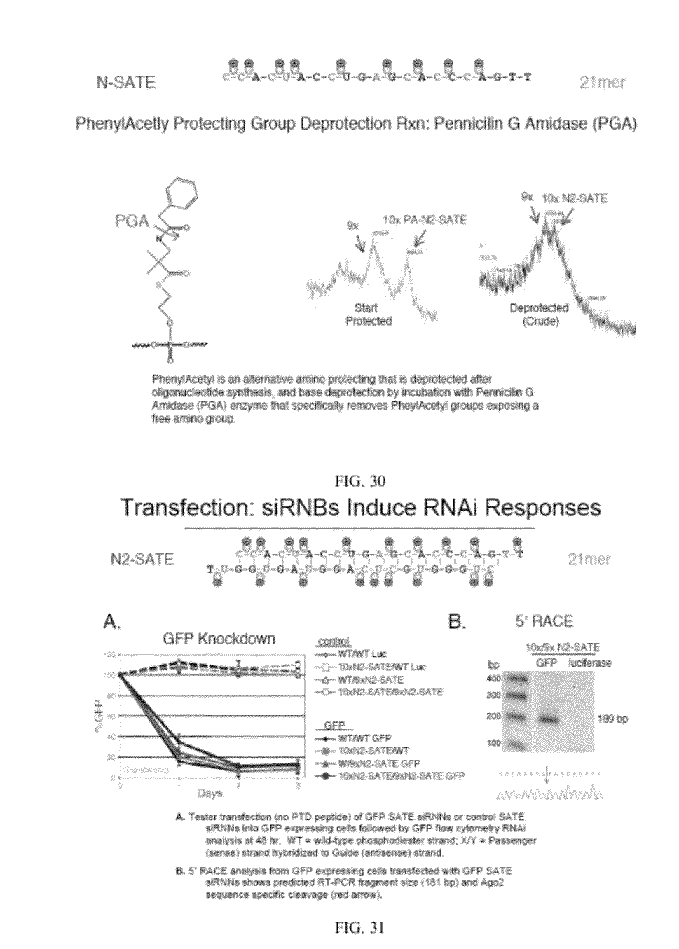

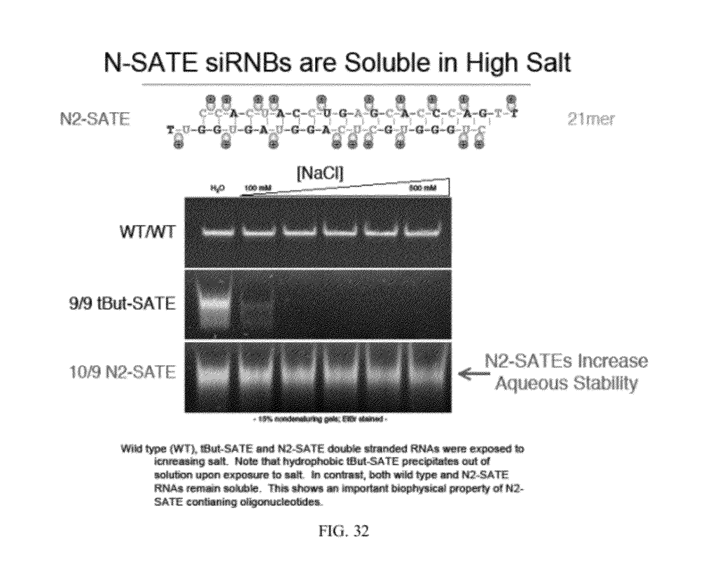

### DETAILED DESCRIPTION

As used herein and in the appended claims, the singular forms “a,” “and,” and “the” include plural referents unless the context clearly dictates otherwise. Thus, for example, reference to “a PTD” includes a plurality of such PTDs and reference to “the cell” includes reference to one or more cells known to those skilled in the art, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

The publications discussed above and throughout the text are provided solely for their disclosure prior to the filing date of the present application. Nothing herein is to be construed as an admission that the inventors are not entitled to antedate such disclosure by virtue of prior disclosure.

The ability to deliver certain bioactive agents to the interior of cells is problematical due to the bioavailability restriction imposed by the cell membrane. The plasma membrane of the cell forms a barrier that restricts the intracellular uptake of molecules to those which are sufficiently non-polar and smaller than approximately 500 daltons in size. Previous efforts to enhance the cellular internalization of proteins have focused on fusing proteins with receptor ligands (Ng et al., Proc. Natl. Acad. Sci. USA, 99:10706-11, 2002) or by packaging them into caged liposomal carriers (Abu-Amer et al., J. Biol. Chem. 276:30499-503, 2001). However, these techniques often result in poor cellular uptake and intracellular sequestration into the endocytic pathway.

Other highly charged nucleic acid molecules with therapeutic potential face the same delivery barrier. For example, RNA aptamers have great potential to bind to, sequester and inhibit proteins, but at >10,000 Daltons and highly charged, they have no or limited ability to enter cells on their own. The methods and compositions of the disclosure allow for intracellular delivery of RNA aptamers, siRNA and DNA vectors.

Due to their anionic charge and large size of ˜14,000 Daltons, delivery of siRNA is a formidable challenge in mammals, including humans. However, cationically charged peptides and proteins have led to advancements in oligonucleotides. For example, linking a protein transduction domains (PTDs) to a nucleic acid has provided some advancement in oligonucleotide delivery. However, even in these instances the anionic and cationic charges of the siRNA and PTD, respectively, often neutralize each other reducing uptake which is promoted by having a cationic charge.

The disclosure provides methods and compositions to facilitate and improve cellular uptake of nucleic acid molecules by protecting/neutralizing the charge associated with an oligonucleotide or polynucleotide. In some embodiments, the compositions of the disclosure provide a cationic charge to the nucleic acid to promote uptake. In other embodiments, an additional cationically charged moiety may be linked to the nucleic acid molecule.

The disclosure provides compositions and methods for the delivery of sequence specific oligonucleotides or polynucleotides useful to selectively treat human diseases and to promote research. The compositions and methods of the disclosure more effectively deliver oligonucleotides and polynucleotides, including siRNAs, RNA aptamers, and DNA vectors to subjects and to cells. The disclosure overcomes size and charge limitations that make such RNAi constructs difficult to deliver or undeliverable. By reversibly neutralizing the anionic charge on a nucleic acids (e.g., dsRNA), a construct comprising a phosphotriester and/or phosphothioate protecting group according to the disclosure can deliver nucleic acids into a cell in vitro and in vivo.

The disclosure provides nucleic acid constructs comprising a charge neutralizing moiety (e.g., a phosphotriester and/or phosphothioate protecting groups). The construct can further include compositions useful in cellular transduction and cellular modulation. Such compositions can include transduction moiety domains comprising a membrane transport function and may further comprise a nucleic acid binding domain sufficient to reversibly neutralize anionic charges on nucleic acids.

As demonstrated herein the addition of one or more removable (e.g., reversibly attached) charge neutralizing moiety to a nucleic acid can effectively facilitate cell transduction. Any nucleic acid, regardless of sequence composition, can be modified by a charge neutralizing moiety of the disclosure.

The disclosure provides oligonucleotides or polynucleotides having, in some embodiments, one or more bioreversible charge neutralizing moieties that contribute to chemical and biophysical properties that enhance cellular membrane penetration and resistance to exo- and endonuclease degradation. The disclosure further provided amidite reagents for the synthesis of the bioreversible protected oligonucleotides or polynucleotides. Moreover, these protecting groups are stable during the synthetic processes.

The oligonucleotides or polynucleotides of the disclosure having one or more bioreversible charge neutralizing moieties are sometimes referred to as pro-oligonucleotides or pro-polynucleotides. In embodiments of this disclosure, the pro-oligonucleotides are capable of improved cellular lipid bilayers penetrating potential as well as resistance to exo- and endonuclease degradation in vivo and in vitro. In cells, the charge neutralizing moieties can be removed by the action of a thio-esterase or by reducing conditions, enzymatic activity (e.g., endogenous carboxyesterases) and the like to yield biologically active oligonucleotide compounds that are capable of hybridizing to and/or having an affinity for specific endogenous nucleic acids.

The charge neutralizing moieties can be used with antisense oligonucleotides of synthetic DNA or RNA or mixed molecules of complementary sequences to a target sequence belonging to a gene or to an RNA messenger whose expression they are specifically designed to block or down-regulate. The antisense oligonucleotides may be directed against a target messenger RNA sequence or, alternatively against a target DNA sequence, and hybridize to the nucleic acid to which they are complementary. Accordingly, these molecules effectively block or down-regulate gene expression.

Charge neutralized oligonucleotides or polynucleotides may also be directed against certain bicatenary DNA regions (homopurine/homopyrimidine sequences or sequences rich in purines/pyrimidines) and thus form triple helices. The formation of a triple helix, at a particular sequence, can block the interaction of protein factors which regulate or otherwise control gene expression and/or may facilitate irreversible damage to be introduced to a specific nucleic acid site if the resulting oligonucleotide is made to possess a reactive functional group.

Provided herein are nucleic acid constructs, and methods of producing such constructs, that can be used for facilitating the delivery of oligonucleotides or polynucleotides in to cells. In one embodiment, a nucleic acid construct includes one or more charge neutralizing moieties to neutralize the phosphodiester anionic charge associated with a nucleic acid, such as RNA and/or DNA. Once inside the cell, the charge neutralizing moiety can be removed from the construct by intracellular processes that include disulfide linkage reduction, ester hydrolysis or other enzyme-mediated processes (e.g., thio-esterase activity). In other embodiments, the nucleic acid construct comprising one or more charge neutralizing moieties further comprises one or more transduction domains such as a protein transduction domain (PTD). For example, a PTD can be conjugated directly to an oligonucleotide (e.g., an RNA or DNA) comprising the nucleic acid construct, such as at the 5′ and/or 3′ end via a free thiol group. For example, a PTD can be linked to the construct by a biologically sensitive and reversible manner, such as a disulfide linkage. This approach can be applied to any oligonucleotide or polynucleotide length and will allow for delivery of RNA (e.g., siRNA, RNA apatmer) or DNA into cells.

In another embodiment, a nucleic acid construct can include a basic group, such as guanidium group (similar to the head group arginine, an active component of the PTD), linked to the reversible protecting group and thereby limit the need for the PTD.

Accordingly, provided herein are nucleotides (e.g., RNA or DNA) synthesized to include a charge neutralizing moiety for the delivery of nucleic acid sequences across a cell membrane. The construct can also include, for example, one or more transduction domains and/or a protecting group that contains a basic group. Once inside the cell the oligonucleotide/polynucleotide of the nucleic acid construct reverts to an unprotected/wild type oligonucleotide/polynucleotide based on the reducing environment, by hydrolysis or other enzymatic activity (e.g., thioesterase activity).

An isolated charge protected oligonucleotide or polynucleotide construct refers to an oligonucleotide or polynucleotide comprising a nucleotide with a charge neutralizing moiety.

The charge-neutralized oligonucleotide can be synthesized using a phosphoramidite structure having the general formula below:

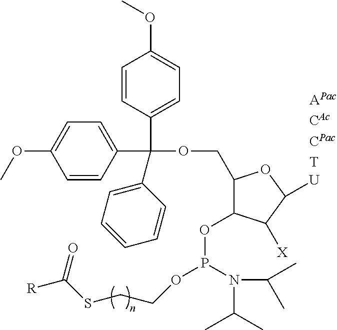

The structure above comprising a U, T, C or A nucleotides can be used in the synthesis of oligonucleotides in an RNA synthesizer.

As used herein an anionic charge neutralizing moiety or group refers to a molecule or chemical group that can reduce the overall net anionic charge of an oligonucleotide or polynucleotide to which it is associated. The amino-S-acyl-thio alkyl moieties as described herein are anionic charge-neutralizing moieties. These charge-neutralizing moieties are reversible. One or more anionic charge-neutralizing moieties or groups can be associated with an oligonucleotide or polynucleotide wherein each independently contributes to a reduction or the anionic charge and or increase in cationic charge of the oligonucleotide or polynucleotide. For example, one or more charge-neutralizing moieties can be associated with an oligonucleotide and the “protected oligonucleotide” associated with one or more cationic transduction domains (e.g., PTDs), such that the overall net anionic charge of the construct is reduced or the overall net charge of the construct is neutral or the overall net charge of the construct is cationic relative to the oligonucleotide without the charge neutralizing moiety and/or PTD.

The disclosure provides charge-neutralizing moieties, nucleotides comprising such charge-neutralizing moieties and oligonucleotides or polynucleotides comprising such charge-neutralizing moieties. In one embodiment, the charge neutralizing moiety has the general formula:

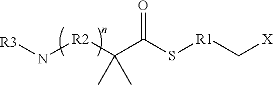

(generally referred to herein as N-SATE) wherein R₁ may or may not be present, when R₁ is present, R₁ is selected from the group consisting of alkyl, substituted alkyl, alkoxy, substituted alkoxy, cycloalkyl, substituted cycloalkyl, alkenyl, substituted alkenyl, alkynyl, substituted alkynyl, aryl, substituted aryl, heterocyclic, or substituted heterocyclic;
wherein R₂ may or may not be present, when R₂ is present, R₂ is selected from the group consisting of a 1 to 7 atom alkyl, substituted alkyl, alkoxy, substituted alkoxy, cycloalkyl, substituted cycloalkyl, alkenyl, substituted alkenyl, alkynyl, substituted alkynyl, aryl, substituted aryl, heterocyclic, or substituted heterocyclic, wherein X is where the charge-neutralizing moiety is linked to the nucleic acid backbone, and
wherein R3 is H, H₂, or a protecting group. In one embodiment, the charge neutralizing moiety (N-SATE) comprises

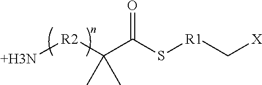

wherein R₁ may or may not be present, when R₁ is present, R₁ is selected from the group consisting of alkyl, substituted alkyl, alkoxy, substituted alkoxy, cycloalkyl, substituted cycloalkyl, alkenyl, substituted alkenyl, alkynyl, substituted alkynyl, aryl, substituted aryl, heterocyclic, or substituted heterocyclic;
wherein R₂ may or may not be present, when R₂ is present, R₂ is selected from the group consisting of a 1 to 7 atom alkyl, substituted alkyl, alkoxy, substituted alkoxy, cycloalkyl, substituted cycloalkyl, alkenyl, substituted alkenyl, alkynyl, substituted alkynyl, aryl, substituted aryl, heterocyclic, or substituted heterocyclic, wherein X is the nucleic acid backbone. In one embodiment, the charge neutralizing moiety is selected from the group consisting of:

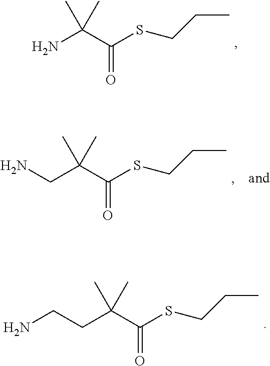

In yet another embodiment, the charge-neutralizing moiety (N-SATE) comprising a protecting group is selected from the following:

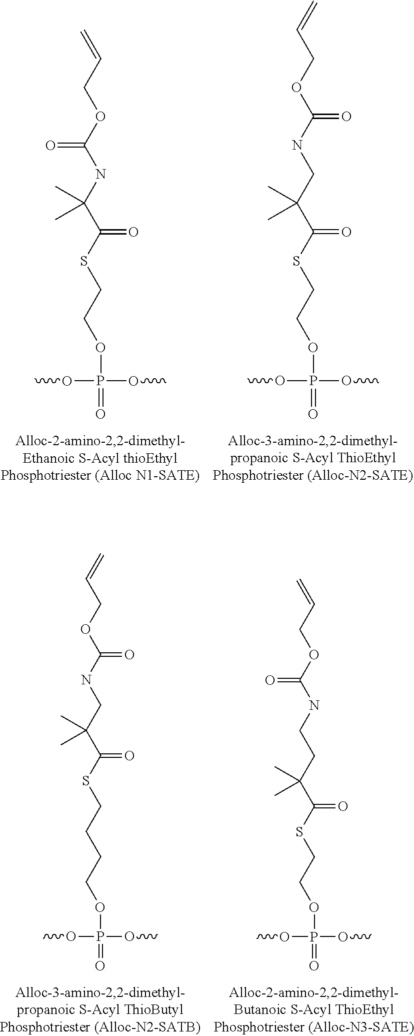

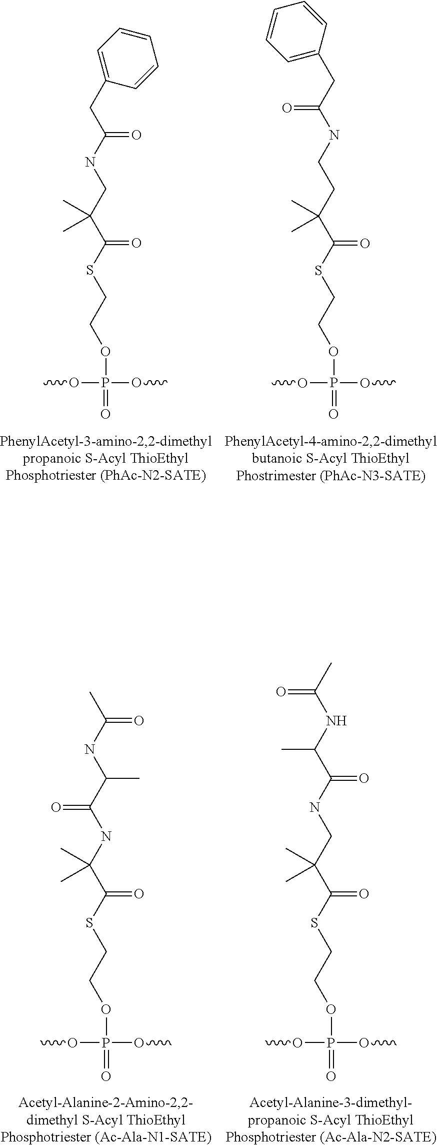

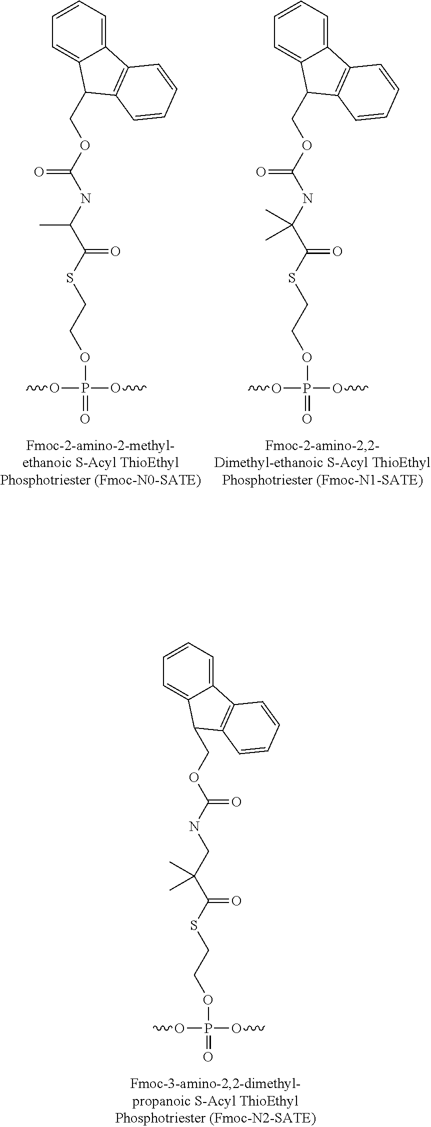

A brief description of various chemical groups are described below. The selection of a group is based upon steric hinderances as will be readily apparent to one of skill in the art. Further, the final structure should have a general cationic charge or be neutral. Again selection of such groups to fit the foregoing criteria will be readily apparent to one of skill the art.

Alkyl groups include straight-chain, branched and cyclic alkyl groups. Alkyl groups include those having from 1 to 20 carbon atoms. Alkyl groups include small alkyl groups having 1 to 3 carbon atoms. Alkyl groups include medium length alkyl groups having from 4-10 carbon atoms. Alkyl groups include long alkyl groups having more than 10 carbon atoms, particularly those having 10-20 carbon atoms. Cyclic alkyl groups include those having one or more rings. Cyclic alkyl groups include those having a 3-, 4-, 5-, 6-, 7-, 8-, 9- or 10-member carbon ring and particularly those having a 3-, 4-, 5-, 6-, or 7-member ring. The carbon rings in cyclic alkyl groups can also carry alkyl groups. Cyclic alkyl groups can include bicyclic and tricyclic alkyl groups. Alkyl groups optionally include substituted alkyl groups. Substituted alkyl groups include among others those which are substituted with aryl groups, which in turn can be optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted.

Alkenyl groups include straight-chain, branched and cyclic alkenyl groups. Alkenyl groups include those having 1, 2 or more double bonds and those in which two or more of the double bonds are conjugated double bonds. Alkenyl groups include those having from 2 to 20 carbon atoms. Alkenyl groups include small alkyl groups having 2 to 3 carbon atoms. Alkenyl groups include medium length alkenyl groups having from 4-10 carbon atoms. Alkenyl groups include long alkenyl groups having more than 10 carbon atoms, particularly those having 10-20 carbon atoms. Cyclic alkenyl groups include those having one or more rings. Cyclic alkenyl groups include those in which a double bond is in the ring or in an alkenyl group attached to a ring. Cyclic alkenyl groups include those having a 3-, 4-, 5-, 6-, 7-, 8-, 9- or 10-member carbon ring and particularly those having a 3-, 4-, 5-, 6- or 7-member ring. The carbon rings in cyclic alkenyl groups can also carry alkyl groups. Cyclic alkenyl groups can include bicyclic and tricyclic alkyl groups. Alkenyl groups are optionally substituted. Substituted alkenyl groups include among others those which are substituted with alkyl or aryl groups, which groups in turn can be optionally substituted. Specific alkenyl groups include ethenyl, prop-1-enyl, prop-2-enyl, cycloprop-1-enyl, but-1-enyl, but-2-enyl, cyclobut-1-enyl, cyclobut-2-enyl, pent-1-enyl, pent-2-enyl, branched pentenyl, cyclopent-1-enyl, hex-1-enyl, branched hexenyl, cyclohexenyl, all of which are optionally substituted.

Aryl groups include groups having one or more 5- or 6-member aromatic or heteroaromatic rings. Aryl groups can contain one or more fused aromatic rings. Heteroaromatic rings can include one or more N, O, or S atoms in the ring. Heteroaromatic rings can include those with one, two or three N, those with one or two O, and those with one or two S. Aryl groups are optionally substituted. Substituted aryl groups include among others those which are substituted with alkyl or alkenyl groups, which groups in turn can be optionally substituted. Specific aryl groups include phenyl groups, biphenyl groups, pyridinyl groups, and naphthyl groups, all of which are optionally substituted.

Arylalkyl groups are alkyl groups substituted with one or more aryl groups wherein the alkyl groups optionally carry additional substituents and the aryl groups are optionally substituted. Specific alkylaryl groups are phenyl-substituted alkyl groups, e.g., phenylmethyl groups.

Alkylaryl groups are aryl groups substituted with one or more alkyl groups wherein the alkyl groups optionally carry additional substituents and the aryl groups are optionally substituted. Specific alkylaryl groups are alkyl-substituted phenyl groups such as methylphenyl.

Rings can be optionally substituted cycloalkyl groups, optionally substituted cycloalkenyl groups or aromatic groups. The rings may contain 3, 4, 5, 6, 7 or more carbons. The rings may be heteroaromatic in which one, two or three carbons in the aromatic ring are replaced with N, O or S. The rings may be heteroalkyl or heteroalkenyl, in which one or more CH₂ groups in the ring are replaced with O, N, NH, or S.

Optional substitution of any alkyl, alkenyl and aryl groups includes substitution with one or more of the following substituents: halogens, —CN, —COOR, —OR, —COR, —OCOOR, —CON(R)₂, —OCON(R)₂, —N(R)₂, —NO₂, —SR, —SO₂R, —SO₂N(R)₂ or —SOR groups. Optional substitution of alkyl groups includes substitution with one or more alkenyl groups, aryl groups or both, wherein the alkenyl groups or aryl groups are optionally substituted. Optional substitution of alkenyl groups includes substitution with one or more alkyl groups, aryl groups, or both, wherein the alkyl groups or aryl groups are optionally substituted. Optional substitution of aryl groups includes substitution of the aryl ring with one or more alkyl groups, alkenyl groups, or both, wherein the alkyl groups or alkenyl groups are optionally substituted.

Optional substituents for alkyl, alkenyl and aryl groups include among others:

—COOR where R is a hydrogen or an alkyl group or an aryl group and more specifically where R is methyl, ethyl, propyl, butyl, or phenyl groups all of which are optionally substituted;

—COR where R is a hydrogen, or an alkyl group or an aryl groups and more specifically where R is methyl, ethyl, propyl, butyl, or phenyl groups all of which groups are optionally substituted;

—CON(R)₂ where each R, independently of each other R, is a hydrogen or an alkyl group or an aryl group and more specifically where R is methyl, ethyl, propyl, butyl, or phenyl groups all of which groups are optionally substituted; R and R can form a ring which may contain one or more double bonds;

—OCON(R)₂ where each R, independently of each other R, is a hydrogen or an alkyl group or an aryl group and more specifically where R is methyl, ethyl, propyl, butyl, or phenyl groups all of which groups are optionally substituted; R and R can form a ring which may contain one or more double bonds;

—N(R)₂ where each R, independently of each other R, is a hydrogen, or an alkyl group, acyl group or an aryl group and more specifically where R is methyl, ethyl, propyl, butyl, or phenyl or acetyl groups all of which are optionally substituted; or R and R can form a ring which may contain one or more double bonds.

—SR, —SO₂R, or —SOR where R is an alkyl group or an aryl groups and more specifically where R is methyl, ethyl, propyl, butyl, phenyl groups all of which are optionally substituted; for —SR, R can be hydrogen;

—OCOOR where R is an alkyl group or an aryl groups; —

—SO₂N(R)₂ where R is a hydrogen, an alkyl group, or an aryl group and R and R can form a ring;

—OR where R═H, alkyl, aryl, or acyl; for example, R can be an acyl yielding —OCOR* where R* is a hydrogen or an alkyl group or an aryl group and more specifically where R* is methyl, ethyl, propyl, butyl, or phenyl groups all of which groups are optionally substituted.

Specific substituted alkyl groups include haloalkyl groups, particularly trihalomethyl groups and specifically trifluoromethyl groups. Specific substituted aryl groups include mono-, di-, tri, tetra- and pentahalo-substituted phenyl groups; mono-, di-, tri-, tetra-, penta-, hexa-, and hepta-halo-substituted naphthalene groups; 3- or 4-halo-substituted phenyl groups, 3- or 4-alkyl-substituted phenyl groups, 3- or 4-alkoxy-substituted phenyl groups, 3- or 4-RCO-substituted phenyl, 5- or 6-halo-substituted naphthalene groups. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups, and methoxyphenyl groups, particularly 4-methoxyphenyl groups.

Where an oligonucleotide or polynucleotide is linked to a PTD, charge neutralization of the anionically charged oligonucleotide or polynucleotide frees the cationically charged PTD to productively interact with the cell surface and also prevents aggregation of the conjugate. For example, the exposed cationic charged PTD interacts with the cell surface and induces macropinocytosis. The oligonucleotide is released into the cytoplasm. Once inside the cell, the charge neutralizing group (e.g., an amino-S-acyl thio alkyl) can be cleaved off by cellular processes, such as a reducing enzyme, oxidizing enzyme, reducing agent, oxidizing agent or esterase, unprotecting the oligonucleotide or polynucleotide allowing the nucleic acid to revert to its natural configuration.

As used herein, a nucleic acid domain, used interchangeably with oligonucleotide or polynucleotide domain, can be any oligonucleotide or polynucleotide (e.g., a ribozyme, antisense molecule, siRNA, dsRNA, polynucleotide, oligonucleotide and the like). Oligonucleotides or polynucleotides generally contain phosphodiester bonds, although in some cases, nucleic acid analogs are included that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g. to increase the stability and half-life of such molecules in physiological environments. Mixtures of naturally occurring nucleic acids and analogs are encompassed by the term oligonucleotide and polynucleotide; alternatively, mixtures of different nucleic acid analogs, and mixtures of naturally occurring nucleic acids and analogs can be made. Furthermore, hybrids of RNN, RNB, DNA, and RNA can be used. dsDNA, ssDNA, dsRNA, siRNA are encompassed by the term oligonucleotide and polynucleotide.

A polynucleotide refers to a polymeric compound made up of any number of covalently bonded nucleotide monomers, including nucleic acid molecules such as DNA and RNA molecules, including single- double- and triple-stranded such molecules, and is expressly intended to embrace that group of polynucleotides commonly referred to as “oligonucleotides”, which are typically distinguished as having a relatively small number (no more than about 30, e.g., about 5-10, 10-20 or 20-30) of nucleotide bases.

As used herein, the term “siRNA” is an abbreviation for “short interfering RNA”, also sometimes known as “small interfering RNA” or “silencing RNA”, and refers to a class of about 19-25 nucleotide-long double-stranded ribonucleic acid molecules that in eukaryotes are involved in the RNA interference (RNAi) pathway that results in post-transcriptional, sequence-specific gene silencing.

The term “dsRNA” is an abbreviation for “double-stranded RNA” and as used herein refers to a ribonucleic acid molecule having two complementary RNA strands and which stands distinct from siRNA in being at least about 26 nucleotides in length, and more typically is at least about 50 to about 100 nucleotides in length.

As described above, the nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus, e.g. the individual units of a peptide nucleic acid, each containing a base, are referred to herein as a nucleoside.

The nucleic acid domain of a nucleic acid construct described herein is not limited by any particular sequence. Any number of oligonucleotide or polynucleotides useful for diagnostics, therapeutics and research can be used in the methods and compositions of the disclosure. Various sources of oligonucleotides and polynucleotides are available to one of skill in the art. For example, fragments of a genome may be isolated and the isolated polynucleotides modified in accordance with the disclosure to reduce the overall net anionic charge using an amino S-acyl thio alkyl charge neutralizing moiety or may be used as a source for extension of the oligonucleotide or polynucleotide using, for example, nucleic acid synthesis techniques known in the art.

The practice of phosphoramidite chemistry to prepare oligonucleotides is known from the published work of M. Caruthers and S. Beaucage and others. U.S. Pat. Nos. 4,458,066, 4,500,707, 5,132,418, 4,415,732, 4,668,777, 4,973,679, 5,278,302, 5,153,319, 5,218,103, 5,268,464, 5,000,307, 5,319,079, 4,659,774, 4,672,110, 4,517,338, 4,725,677 and Re. 34,069, each of which is herein incorporated by reference, describe methods of oligonucleotide synthesis. Additionally, the practice of phosphoramidite chemistry has been systematically reviewed by Beaucage and Iyer in Beaucage, S. L. and Iyer, R. P., Tetrahedron, 1992, 48, 2223-2311 and Beaucage, S. L. and Iyer, R. P., Tetrahedron, 1993, 49, 6123-6194, or references referred to therein, all of which are herein incorporated by reference.

Nucleic acid synthesizers are commercially available and their use is generally understood by persons of ordinary skill in the art as being effective in generating nearly any oligonucleotide of reasonable length which may be desired.

In practicing phosphoramidite chemistry useful 5′OH sugar blocking groups are trityl, monomethoxytrityl, dimethoxytrityl and trimethoxytrityl, especially dimethoxytrityl (DMTr). In practicing phosphoramidite chemistry useful phosphite activating groups, i.e., NR₂, are dialkyl substituted nitrogen groups and nitrogen heterocycles. One approach includes the use of the di-isopropylamino activating group.

Oligonucleotides can be synthesized by a Mermade-6 solid phase automated oligonucleotide synthesizer or any commonly available automated oligonucleotide synthesizer. Triester, phosphoramidite, or hydrogen phosphonate coupling chemistries described in, for example, M. Caruthers, Oligonucleotides: Antisense Inhibitors of Gene Expression., pp. 7-24, J. S. Cohen, ed. (CRC Press, Inc. Boca Raton, Fla., 1989) or Oligonucleotide synthesis, a practical approach, Ed. M. J. Gait, IRL Press, 1984; “Oligonucleotides and Analogues, A Practical Approach”, Ed. F. Eckstein, IRL Press, 1991, are employed by these synthesizers to provide the desired oligonucleotides. The Beaucage reagent, as described in, for example, Journal of American Chemical Society, 1990, 112, 1253-1255, or elemental sulfur, as described in Beaucage et al., Tetrahedron Letters, 1981, 22, 1859-1862, is used with phosphoramidite or hydrogen phosphonate chemistries to provide substituted phosphorothioate oligonucleotides. For example, the reagents comprising the protecting groups recited herein can be used in numerous applications where protection is desired. Such applications include, but are not limited to, both solid phase and solution phase, oligo-synthesis, polynucleotide synthesis and the like. The use of nucleoside and nucleotide analogs is also contemplated by this disclosure to provide oligonucleotide or oligonucleotide analogs bearing the protecting groups disclosed herein. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into an oligonucleotide or oligonucleotide sequence, they allow hybridization with a naturally occurring oligonucleotide sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

For instance, structural groups are optionally added to the ribose or base of a nucleoside for incorporation into an oligonucleotide, such as a methyl, propyl or allyl group at the 2′-0 position on the ribose, or a fluoro group which substitutes for the 2′-O group, or a bromo group on the ribonucleoside base. For use with phosphoramidite chemistry, various amidite reagents are commercially available, including 2′-deoxy amidites, 2′-O-methyl amidites and 2′-O-hydroxyl amidites. Any other means for such synthesis may also be employed. The actual synthesis of the oligonucleotides is well within the talents of those skilled in the art. It is also well known to use similar techniques to prepare other oligonucleotides such as the phosphorothioates, methyl phosphonates and alkylated derivatives. It is also well known to use similar techniques and commercially available modified amidites and controlled-pore glass (CPG) products such as biotin, Cy3, fluorescein, acridine or psoralen-modified amidites and/or CPG (available from Glen Research, Sterling Va.) to synthesize fluorescently labeled, biotinylated or other conjugated oligonucleotides.

Although the phosphotriester neutralizing/protecting groups described herein are useful for neutralizing the anionic charge of a nucleic acid domain, additional cationically charged moieties linked to a protected nucleic acid domain can be used to further facilitate uptake of oligonucleotide and polynucleotides. The recent discovery of several proteins which could efficiently pass through the plasma membrane of eukaryotic cells has led to the identification of a novel class of proteins from which peptide transduction domains have been derived.

For example, charge neutralization of anionic nucleic acid (e.g., an RNA molecule) using an amino S-acyl thio alkyl moiety (SATE) promotes uptake. In embodiments where the charge neutralized anionic nucleic acid is linked to a PTD the charge neutralization of the anionic charged nucleic acid frees the cationic PTD to traverse the membrane as well as prevents aggregation of the conjugate due to a net cationic charge. The exposed free cationic charge of the PTD can then effectively interact with a cell surface, induce macropinocytosis and escape from the macropinosome into the cytoplasm. Once inside a cell, the phosphotriester and/or phosphothioate protecting group(s) can be removed by intracellular processes, such as reduction of a disulfide linkage or ester hydrolysis, allowing for removal from the construct in the cytoplasm. In one embodiment, the removal of the charge neutralizing group is through the activity of a thio-esterase. A nucleic acid construct that includes, for example, dsRNA can then be hydrolyzed by Dicer, an RNAse III-like ribonuclease, thereby releasing siRNA that silences a target gene.

A number of protein transduction domains/peptides are known in the art and have been demonstrated to facilitate uptake of heterologous molecules linked to the transdomain (e.g., cargo molecules). Such transduction domains facilitate uptake through a process referred to as macropinocytosis. Macropinocytosis is a nonselective form of endocytosis that all cells perform.

The best characterized of these proteins are the Drosophila homeoprotein antennapedia transcription protein (AntHD) (Joliot et al., New Biol. 3:1121-34, 1991; Joliot et al., Proc. Natl. Acad. Sci. USA, 88:1864-8, 1991; Le Roux et al., Proc. Natl. Acad. Sci. USA, 90:9120-4, 1993), the herpes simplex virus structural protein VP22 (Elliott and O'Hare, Cell 88:223-33, 1997), the HIV-1 transcriptional activator TAT protein (Green and Loewenstein, Cell 55:1179-1188, 1988; Frankel and Pabo, Cell 55:1189-1193, 1988), and more recently the cationic N-terminal domain of prion proteins. Not only can these proteins pass through the plasma membrane but the attachment of other proteins, such as the enzyme β-galactosidase, was sufficient to stimulate the cellular uptake of these complexes. Such chimeric proteins are present in a biologically active form within the cytoplasm and nucleus. Characterization of this process has shown that the uptake of these fusion polypeptides is rapid, often occurring within minutes, in a receptor independent fashion. Moreover, the transduction of these proteins does not appear to be affected by cell type and can efficiently transduce 100% of cells in culture with no apparent toxicity (Nagahara et al., Nat. Med. 4:1449-52, 1998). In addition to full-length proteins, protein transduction domains have also been used successfully to induce the intracellular uptake of DNA (Abu-Amer, supra), antisense oligonucleotides (Astriab-Fisher et al., Pharm. Res, 19:744-54, 2002), small molecules (Polyakov et al., Bioconjug. Chem. 11:762-71, 2000) and even inorganic 40 nanometer iron particles (Dodd et al., J. Immunol. Methods 256:89-105, 2001; Wunderbaldinger et al., Bioconjug. Chem. 13:264-8, 2002; Lewin et al., Nat. Biotechnol. 18:410-4, 2000; Josephson et al., Bioconjug., Chem. 10:186-91, 1999) suggesting that there is no apparent size restriction to this process. The effective transduction using transduction domains is, in part, limited by the overall molecular charge on the PTD-cargo construct.

The fusion of a protein transduction domain (PTD) with a heterologous molecule (e.g., a polynucleotide, small molecule, or protein) is sufficient to cause their transduction into a variety of different cells in a concentration-dependent manner. Moreover, this technique for protein delivery appears to circumvent many problems associated with DNA and drug based techniques.

PTDs are typically cationic in nature. These cationic protein transduction domains track into lipid raft endosomes carrying with them their linked cargo and release their cargo into the cytoplasm by disruption of the endosomal vesicle. In general, the transduction domain of a nucleic acid construct of the disclosure can be nearly any synthetic or naturally-occurring amino acid sequence that can transduce or assist in the transduction of the fusion molecule. Typically, the transduction domain is cationically charged. For example, transduction can be achieved in accord with the disclosure by use of a nucleic acid construct including phosphotriester and/or phosphothioate protecting groups and a protein sequence such as an HIV TAT protein or fragment thereof that is linked at the N-terminal or C-terminal end to an oligonucleotide or polynucleotide comprising a phosphotriester and/or phosphothioate protecting group. In some embodiments, the nucleic acid may comprise a phosphotriester and/or phosphothioate protecting group and may also comprise a double stranded RNA binding domain (e.g., a DRBD). The transducing protein domain, for example, can be the Antennapedia homeodomain or the HSV VP22 sequence, the N-terminal fragment of a prion protein or suitable transducing fragments thereof such as those known in the art.

The type and size of the PTD that can be linked to a charge-neutralized nucleic acid molecule will be guided by several parameters including the extent of transduction desired. PTDs will be capable of transducing at least about 20%, 25%, 50%, 75%, 80%, 90%, 95%, 98% 99% or 100% of the cells. Transduction efficiency, typically expressed as the percentage of transduced cells, can be determined by several conventional methods.

PTDs will manifest cell entry and exit rates (sometimes referred to as k₁ and k₂, respectively) that favor at least picomolar amounts of the fusion molecule in the cell. The entry and exit rates of the PTD and any cargo can be readily determined, or at least approximated, by standard kinetic analysis using detectably-labeled fusion molecules. Typically, the ratio of the entry rate to the exit rate will be in the range of between about 5 to about 100 up to about 1000.

In one embodiment, a PTD useful in the methods and compositions of the disclosure comprise a peptide featuring substantial alpha-helicity. It has been discovered that transduction is optimized when the PTD exhibits significant alpha-helicity. In another embodiment, the PTD comprises a sequence containing basic amino acid residues that are substantially aligned along at least one face of the peptide. A PTD domain of the disclosure may be a naturally occurring peptide or a synthetic peptide.

In one embodiment of the disclosure, the PTD comprises an amino acid sequences comprising a strong alpha helical structure with arginine (Arg) residues down the helical cylinder. In yet another embodiment, the PTD domain comprises a peptide represented by the following general formula: B₁—X₁—X₂—X₃—B₂—X₄—X₅—B₃ (SEQ ID NO:1) wherein B₁, B₂, and B₃ are each independently a basic amino acid, the same or different; and X₁, X₂, X₃, X₄ and X₅ are each independently an alpha-helix enhancing amino acid, the same or different. In another embodiment, the PTD domain is represented by the following general formula: B₁—X₁—X₂—B₂—B₃—X₃—X₄—B₄ (SEQ ID NO:2) wherein B₁, B₂, B₃, and B₄ are each independently a basic amino acid, the same or different; and X₁, X₂, X₃, and X₄ are each independently an alpha-helix enhancing amino acid the same or different.

Additionally PTD domains comprise basic residues, e.g., lysine (Lys) or arginine (Arg), and further including at least one proline (Pro) residue sufficient to introduce “kinks” into the domain. Examples of such domains include the transduction domains of prions. For example, such a peptide comprises KKRPKPG (SEQ ID NO:3).

In one embodiment, the domain is a peptide represented by the following sequence: X—X—R—X—(P/X)—(B/X)—B—(P/X)—X—B-(B/X) (SEQ ID NO:4), wherein X is any alpha helical promoting residue such as alanine; P/X is either proline or X as previously defined; B is a basic amino acid residue, e.g., arginine (Arg) or lysine (Lys); R is arginine (Arg) and B/X is either B or X as defined above.

In another embodiment the PTD is cationic and consists of between 7 and 10 amino acids and has the formula K—X₁—R—X₂—X₁ (SEQ ID NO:5) wherein X₁ is R or K and X₂ is any amino acid. An example of such a peptide comprises RKKRRQRRR (SEQ ID NO:6).

Additional transducing domains include a TAT fragment that comprises at least amino acids 49 to 56 of TAT up to about the full-length TAT sequence (see, e.g., SEQ ID NO:7). A TAT fragment may include one or more amino acid changes sufficient to increase the alpha-helicity of the fragment. In some instances, the amino acid changes introduced will involve adding a recognized alpha-helix enhancing amino acid. Alternatively, the amino acid changes will involve removing one or more amino acids from the TAT fragment that impede alpha helix formation or stability. In a more specific embodiment, the TAT fragment will include at least one amino acid substitution with an alpha-helix enhancing amino acid. Typically a TAT fragment or other PTD will be made by standard peptide synthesis techniques although recombinant DNA approaches may be used in some cases.

Additional transduction proteins (PTDs) that can be used in the nucleic acid constructs of the disclosure include the TAT fragment in which the TAT 49-56 sequence has been modified so that at least two basic amino acids in the sequence are substantially aligned along at least one face of the TAT fragment. Illustrative TAT fragments include at least one specified amino acid substitution in at least amino acids 49-56 of TAT which substitution aligns the basic amino acid residues of the 49-56 sequence along at least one face of the segment and typically the TAT 49-56 sequence.

Additional transduction proteins include the TAT fragment in which the TAT 49-56 sequence includes at least one substitution with an alpha-helix enhancing amino acid. In one embodiment, the substitution is selected so that at least two basic amino acid residues in the TAT fragment are substantially aligned along at least one face of that TAT fragment. In a more specific embodiment, the substitution is chosen so that at least two basic amino acid residues in the TAT 49-56 sequence are substantially aligned along at least one face of that sequence.

Additional examples of PTDs include AntHD, TAT, VP22, cationic prion protein domains, poly-Arg, AGRKKRRQRRR (SEQ ID NO:14), YARKARRQARR (SEQ ID NO:15), YARAAARQARA (SEQ ID NO:16), YARAARRAARR (SEQ ID NO:17), YARAARRAARA (SEQ ID NO:18), YARRRRRRRRR (SEQ ID NO:19), YAAARRRRRRR (SEQ ID NO:20) and functional fragments and variants thereof. The disclosure provides, in one embodiment, methods and compositions that combine the use of PTDs such as TAT and poly-Arg, with a charge neutralized nucleic acids. By charge neutralized is meant that the overall anionic charge of the nucleic acid (e.g., oligonucleotide or polynucleotide) is reduced in the construct, neutralized or more cationic than the same nucleic acid in the absence of a phosphotriester and/or phosphothioate protecting group or a phosphotriester and/or phosphothioate protecting group and a binding domain and/or a protein transduction domain capable of neutralizing the anionic charge on a nucleic acid (i.e., the “cargo”) domain.

Also included are chimeric PTD domains. Such chimeric transducing proteins include parts of at least two different transducing proteins. For example, chimeric transducing proteins can be formed by fusing two different TAT fragments, e.g., one from HIV-1 and the other from HIV-2 or one from a prion protein and one from HIV.

PTDs can be linked or fused with any number of other molecules including an oligonucleotide or polynucleotide. Alternatively, the nucleic acid construct or PTD can be bound to other molecular entities including nucleic acid binding domains, targeting moieties and the like. For example, two or more PTDs (e.g., 1-5, 2-4, typically 3) can be linked in series or separated by one or more other domains (e.g., a nucleic acid domain or peptide linkers). A nucleic acid binding domain can promote uptake of a fusion construct comprising a nucleic acid (including an oligonucleotide or polynucleotide comprising a protecting group) by reducing the anionic charge such that the cationic charge of the PTD domain is sufficient to transduce/traverse a cell's membrane. It will be understood that the PTD may be fused to an oligonucleotide or polynucleotide comprising an anionic charge neutralizing group and may further be linked to a nucleic acid binding domain. Exemplary RNA binding proteins (e.g., DRBD) include histone, RDE-4 protein, or protamine. Additional dsRNA binding proteins (and their Accession numbers in parenthesis) include: PKR (AAA36409, AAA61926, Q03963), TRBP (P97473, AAA36765), PACT (AAC25672, AAA49947, NP609646), Staufen (AAD17531, AAF98119, AAD17529, P25159), NFAR1 (AF167569), NFAR2 (AF167570, AAF31446, AAC71052, AAA19960, AAA19961, AAG22859), SPNR (AAK20832, AAF59924, A57284), RHA (CAA71668, AAC05725, AAF57297), NREBP (AAK07692, AAF23120, AAF54409, T33856), kanadaptin (AAK29177, AAB88191, AAF55582, NP499172, NP198700, BAB19354), HYL1 (NP563850), hyponastic leaves (CAC05659, BAB00641), ADAR1 (AAB97118, P55266, AAK16102, AAB51687, AF051275), ADAR2 P78563, P51400, AAK17102, AAF63702), ADAR3 (AAF78094, AAB41862, AAF76894), TENR (XP059592, CAA59168), RNaseIII (AAF80558, AAF59169, Z81070Q02555/S55784, PO5797), and Dicer (BAA78691, AF408401, AAF56056, S44849, AAF03534, Q9884), RDE-4 (AY071926), FLJ20399 (NP060273, BAB26260), CG1434 (AAF48360, EAA12065, CAA21662), CG13139 (XP059208, XP143416, XP110450, AAF52926, EEA14824), DGCRK6 (BAB83032, XP110167) CG1800 (AAF57175, EAA08039), FLJ20036 (AAH22270, XP134159), MRP-L45 (BAB14234, XP129893), CG2109 (AAF52025), CG12493 (NP647927), CG10630 (AAF50777), CG17686 (AAD50502), T22A3.5 (CAB03384) and Accession number EAA14308.

Peptide linkers that can be used in the fusion polypeptides and methods of the disclosure will typically comprise up to about 20 or 30 amino acids, commonly up to about 10 or 15 amino acids, and still more often from about 1 to 5 amino acids. The linker sequence is generally flexible so as not to hold the fusion molecule in a single rigid conformation. The linker sequence can be used, e.g., to space the PTD domain from the nucleic acid binding domain and/or nucleic acid domain. For example, the peptide linker sequence can be positioned to provide molecular flexibility. The length of the linker moiety is chosen to optimize the biological activity of the polypeptide comprising a PTD domain fusion construct and can be determined empirically without undue experimentation. The linker moiety should be long enough and flexible enough to allow a PTD to freely interact with a nucleic acid or vice versa. Examples of linker moieties are -Gly-Gly—, GGGGS (SEQ ID NO:8), (GGGGS)_{N} (SEQ ID NO:8, repeated), GKSSGSGSESKS (SEQ ID NO:9), GSTSGSGKSSEGKG (SEQ ID NO:10), GSTSGSGKSSEGSGSTKG (SEQ ID NO:11), GSTSGSGKPGSGEGSTKG (SEQ ID NO:12), or EGKSSGSGSESKEF (SEQ ID NO:13). Linking moieties are described, for example, in Huston et al., Proc. Nat'l Acad. Sci. 85:5879, 1988; Whitlow et al., Protein Engineering 6:989, 1993; and Newton et al., Biochemistry 35:545, 1996. Other suitable peptide linkers are those described in U.S. Pat. Nos. 4,751,180 and 4,935,233, which are hereby incorporated by reference.

The methods, compositions, and fusion polypeptides of the disclosure provide enhanced uptake and release of nucleic acid molecules by cells both in vitro and in vivo.

The term “therapeutic” is used in a generic sense and includes treating agents, prophylactic agents, and replacement agents. Examples of therapeutic molecules include, but are not limited to, cell cycle control agents; agents which inhibit cyclin proteins, such as antisense polynucleotides to the cyclin G1 and cyclin D1 genes; dsRNA that can be cleaved to provide siRNA molecules directed to specific growth factors such as, for example, epidermal growth factor (EGF), vascular endothelial growth factor (VEGF), erythropoietin, G-CSF, GM-CSF, TGF-α, TGF-β, and fibroblast growth factor; cytokines, including, but not limited to, Interleukins 1 through 13 and tumor necrosis factors; anticoagulants, anti-platelet agents; TNF receptor domains etc.

Using such methods and compositions, various diseases and disorders can be treated. For example, growth of tumor cells can be inhibited, suppressed, or destroyed upon delivery of an anti-tumor siRNA.

Thus, it is to be understood that the disclosure is not to be limited to any particular transduction domain or oligonucleotide/polynucleotide. Any anionically charged nucleic acid (e.g., dsRNA, siRNA and the like) can be delivered using the methods and compositions of the disclosure.

The polypeptides used in the disclosure (e.g., with respect to particular domains of a fusion polypeptide or the full length fusion polypeptide) can comprise either the L-optical isomer or the D-optical isomer of amino acids or a combination of both. Polypeptides that can be used in the disclosure include modified sequences such as glycoproteins, retro-inverso polypeptides, D-amino acid modified polypeptides, and the like. A polypeptide includes naturally occurring proteins, as well as those which are recombinantly or synthetically synthesized. “Fragments” are a portion of a polypeptide. The term “fragment” refers to a portion of a polypeptide which exhibits at least one useful epitope or functional domain. The term “functional fragment” refers to fragments of a polypeptide that retain an activity of the polypeptide. For example, a functional fragment of a PTD includes a fragment which retains transduction activity.

In some embodiments, retro-inverso peptides are used. “Retro-inverso” means an amino-carboxy inversion as well as enantiomeric change in one or more amino acids (i.e., levantory (L) to dextrorotary (D)). A polypeptide of the disclosure encompasses, for example, amino-carboxy inversions of the amino acid sequence, amino-carboxy inversions containing one or more D-amino acids, and non-inverted sequence containing one or more D-amino acids. Retro-inverso peptidomimetics that are stable and retain bioactivity can be devised as described by Brugidou et al. (Biochem. Biophys. Res. Comm. 214(2): 685-693, 1995) and Chorev et al. (Trends Biotechnol. 13(10): 438-445, 1995).

The disclosure also provides polynucleotides encoding a fusion protein construct of the disclosure. Such polynucleotides comprise sequences encoding one or more PTD domains, and/or a nucleic acid binding domain (e.g., DRBD). The polynucleotide may also encode linker domains that separate one or more of the PTDs and/or nucleic acid binding domains. In one aspect a fusion polypeptide comprising two or more PTD domains is produced and then linked to a charge reduced/protected oligonucleotide or polynucleotide comprising an N-SATE.

A polynucleotide construct can be incorporated (i.e., cloned) into an appropriate vector. For purposes of expression, the polynucleotide encoding a fusion polypeptide of the disclosure may be inserted into a recombinant expression vector. The term “recombinant expression vector” refers to a plasmid, virus, or other vehicle known in the art that has been manipulated by insertion or incorporation of a polynucleotide encoding a fusion polypeptide of the disclosure. The expression vector typically contains an origin of replication, a promoter, as well as specific genes that allow phenotypic selection of the transformed cells. Vectors suitable for such use include, but are not limited to, the T7-based expression vector for expression in bacteria (Rosenberg et al., Gene, 56:125, 1987), the pMSXND expression vector for expression in mammalian cells (Lee and Nathans, J. Biol. Chem., 263:3521, 1988), baculovirus-derived vectors for expression in insect cells, cauliflower mosaic virus, CaMV, and tobacco mosaic virus, TMV, for expression in plants.

Depending on the vector utilized, any of a number of suitable transcription and translation elements (regulatory sequences), including constitutive and inducible promoters, transcription enhancer elements, transcription terminators, and the like may be used in the expression vector (see, e.g., Bitter et al., Methods in Enzymology, 153:516-544, 1987). These elements are well known to one of skill in the art.

The term “operably linked” and “operably associated” are used interchangeably herein to broadly refer to a chemical or physical coupling of two otherwise distinct domains that each have independent biological function. For example, operably linked refers to the functional linkage between a regulatory sequence and the polynucleotide regulated by the regulatory sequence. In another aspect, operably linked refers to the association of a nucleic acid domain and a transduction domain such that each domain retains its independent biological activity under appropriate conditions. Operably linked further refers to the link between encoded domains of the fusion polypeptides such that each domain is linked in-frame to give rise to the desired polypeptide sequence.

In yeast, a number of vectors containing constitutive or inducible promoters may be used (see, e.g., Current Protocols in Molecular Biology, Vol. 2, Ed. Ausubel et al., Greene Publish. Assoc. & Wiley Interscience, Ch. 13, 1988; Grant et al., “Expression and Secretion Vectors for Yeast,” in Methods in Enzymology, Eds. Wu & Grossman, Acad. Press, N.Y., Vol. 153, pp. 516-544, 1987; Glover, DNA Cloning, Vol. II, IRL Press, Wash., D.C., Ch. 3, 1986; “Bitter, Heterologous Gene Expression in Yeast,” Methods in Enzymology, Eds. Berger & Kimmel, Acad. Press, N.Y., Vol. 152, pp. 673-684, 1987; and The Molecular Biology of the Yeast Saccharomyces, Eds. Strathern et al., Cold Spring Harbor Press, Vols. I and II, 1982). A constitutive yeast promoter, such as ADH or LEU2, or an inducible promoter, such as GAL, may be used (“Cloning in Yeast,” Ch. 3, R. Rothstein In: DNA Cloning Vol. 11, A Practical Approach, Ed. DM Glover, IRL Press, Wash., D.C., 1986). Alternatively, vectors may be used which promote integration of foreign DNA sequences into the yeast chromosome.

An expression vector can be used to transform a host cell. By “transformation” is meant a permanent genetic change induced in a cell following incorporation of a polynucleotide exogenous to the cell. Where the cell is a mammalian cell, a permanent genetic change is generally achieved by introduction of the polynucleotide into the genome of the cell. By “transformed cell” or “recombinant host cell” is meant a cell into which (or into an ancestor of which) has been introduced, by means of molecular biology techniques, a polynucleotide encoding a fusion polypeptide of the disclosure. Transformation of a host cell may be carried out by conventional techniques as are known to those skilled in the art. Where the host is prokaryotic, such as E. coli, competent cells which are capable of polynucleotide uptake can be prepared from cells harvested after exponential growth phase and subsequently treated by the CaCl₂ method by procedures known in the art. Alternatively, MgCl₂ or RbCl can be used. Transformation can also be performed after forming a protoplast of the host cell or by electroporation.

A fusion polypeptide of the disclosure can be produced by expression of polynucleotide encoding a fusion polypeptide in prokaryotes. These include, but are not limited to, microorganisms, such as bacteria transformed with recombinant bacteriophage DNA, plasmid DNA, or cosmid DNA expression vectors encoding a fusion polypeptide of the disclosure. The constructs can be expressed in E. coli in large scale. Purification from bacteria is simplified when the sequences include tags for one-step purification by nickel-chelate chromatography. Thus, a polynucleotide encoding a fusion polypeptide can also comprise a tag to simplify isolation of the fusion polypeptide. For example, a polyhistidine tag of, e.g., six histidine residues, can be incorporated at the amino terminal end of the fusion polypeptide. The polyhistidine tag allows convenient isolation of the protein in a single step by nickel-chelate chromatography. A fusion polypeptide of the disclosure can also be engineered to contain a cleavage site to aid in protein recovery the cleavage site may be part of a linker moiety as discussed above. A DNA sequence encoding a desired peptide linker can be inserted between, and in the same reading frame as, a polynucleotide encoding a PTD, or fragment thereof followed by a nucleic acid binding domain, the PTD may also be linked to a desired nucleic acid (e.g., dsRNA, DNA, siRNA, and the like), using any suitable conventional technique. For example, a chemically synthesized oligonucleotide encoding the linker can be ligated between two coding polynucleotides. In particular embodiments, a polynucleotide of the disclosure will encode a fusion polypeptide comprising from two to four separate domains (e.g., one or more PTD domain and one or more a nucleic acid domains) separated by linkers. In some embodiments, once purified, a fusion polypeptide comprising a plurality of PTDs is associated or linked with an oligonucleotide comprising an anionic charge neutralizing group or other anionic charge reducing group.

When the host cell is a eukaryotic cell, such methods of transfection of DNA as calcium phosphate co-precipitates, conventional mechanical procedures, such as microinjection, electroporation, insertion of a plasmid encased in liposomes, or virus vectors may be used. Eukaryotic cells can also be cotransfected with a polynucleotide encoding the PTD-fusion polypeptide of the disclosure, and a second polynucleotide molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the fusion polypeptide (see, e.g., Eukaryotic Viral Vectors, Cold Spring Harbor Laboratory, Gluzman ed., 1982).

Eukaryotic systems, and typically mammalian expression systems, allow for proper post-translational modifications of expressed mammalian proteins to occur. Eukaryotic cells that possess the cellular machinery for proper processing of the primary transcript, glycosylation, phosphorylation, and advantageously secretion of the fusion product can be used as host cells for the expression of the PTD-fusion polypeptide of the disclosure. Such host cell lines may include, but are not limited to, CHO, VERO, BHK, HeLa, COS, MDCK, Jurkat, HEK-293, and WI38.

For long-term, high-yield production of recombinant proteins, stable expression is used. Rather than using expression vectors that contain viral origins of replication, host cells can be transformed with the cDNA encoding a fusion polypeptide of the disclosure controlled by appropriate expression control elements (e.g., promoter, enhancer, sequences, transcription terminators, polyadenylation sites, and the like), and a selectable marker. The selectable marker in the recombinant plasmid confers selectivity (e.g., by cytotoxin resistance) and allows cells to stably integrate the plasmid into their chromosomes and grow to form foci that, in turn, can be cloned and expanded into cell lines. For example, following the introduction of foreign DNA, engineered cells may be allowed to grow for 1-2 days in an enriched media, and then are switched to a selective media. A number of selection systems may be used, including, but not limited to, the herpes simplex virus thymidine kinase (Wigler et al., Cell, 11:223, 1977), hypoxanthine-guanine phosphoribosyltransferase (Szybalska & Szybalski, Proc. Natl. Acad. Sci. USA, 48:2026, 1962), and adenine phosphoribosyltransferase (Lowy et al., Cell, 22:817, 1980) genes can be employed in tk-, hgprt- or aprt-cells, respectively. Also, antimetabolite resistance can be used as the basis of selection for dhfr, which confers resistance to methotrexate (Wigler et al., Proc. Natl. Acad. Sci. USA, 77:3567, 1980; O'Hare et al., Proc. Natl. Acad. Sci. USA, 8:1527, 1981); gpt, which confers resistance to mycophenolic acid (Mulligan & Berg, Proc. Natl. Acad. Sci. USA, 78:2072, 1981; neo, which confers resistance to the aminoglycoside G-418 (Colberre-Garapin et al., J. Mol. Biol., 150:1, 1981); and hygro, which confers resistance to hygromycin genes (Santerre et al., Gene, 30:147, 1984). Additional selectable genes have been described, namely trpB, which allows cells to utilize indole in place of tryptophan; hisD, which allows cells to utilize histinol in place of histidine (Hartman & Mulligan, Proc. Natl. Acad. Sci. USA, 85:8047, 1988); and ODC (ornithine decarboxylase), which confers resistance to the ornithine decarboxylase inhibitor, 2-(difluoromethyl)-DL-ornithine, DFMO (McConlogue L., In: Current Communications in Molecular Biology, Cold Spring Harbor Laboratory, ed., 1987).

Techniques for the isolation and purification of either microbially or eukaryotically expressed PTD-fusion polypeptides of the disclosure may be by any conventional means, such as, for example, preparative chromatographic separations and immunological separations, such as those involving the use of monoclonal or polyclonal antibodies or antigen.

The fusion polypeptides of the disclosure are useful for the delivery of anionically charged nucleic acid molecules (e.g., dsRNA, siRNA, DNA, antisense, ribozymes and the like) for the treatment and/or diagnosis of a number of diseases and disorders. For example, the fusion polypeptides can be used in the treatment of cell proliferative disorders, wherein the protected oligo- or polynucleotide is reversibly modified such that it traverses a cell membrane alone or in associate with a PTD to target genes that induce cell proliferation. The PTD domain increases the overall net cationic charge or reduces the overall net anionic charge of the nucleic acid construct facilitating facilitates uptake by the cell. Thus, the constructs are useful for treatment of cells having cell proliferative disorders. Similarly, the constructs of the disclosure can be used to treat inflammatory diseases and disorders, infections, vascular disease and disorders and the like.

In one embodiment, the construct of the disclosure may alternatively comprise, or in addition to, the PTD, a targeting domain. The targeting domain can be a receptor, receptor ligand or antibody useful for directing the construct to a particular cell type that expresses the cognate binding domain.

Thus, the disclosure provides oligonucleotides comprising N-SATE moieties that reduce the anionic charge (charge neutralized oligonucleotides). The disclosure also provides charge-neutralized oligonucleotides linked to a PTD including PTDs comprising a fusion protein. The disclosure also provides charge-neutralized oligonucleotides comprising an RNA binding domain protein. In some embodiment, combinations of PTDs and RNA binding domain proteins are linked or constructed with a charge-neutralized oligonucleotides. Generally such charge neutralized oligonucleotides and constructs have a (i) a reduced anionic charge, (ii) a neutral charge, or (iii) a cationic charge.

Delivery of a polynucleotide of the disclosure can be achieved by contacting a cell with a polynucleotide using a variety of methods known to those of skill in the art. Because a polynucleotide or oligonucleotide comprising an N-SATE alone or with a PTD has a general neutral or cationic charge the polynucleotide is capable of traversing the cell membrane. In some embodiments, the oligonucleotide is formulated with various carriers, dispersion agents and the like, as described more fully elsewhere herein.

Typically a construct of the disclosure will be formulated with a pharmaceutically acceptable carrier, although the fusion polypeptide may be administered alone, as a pharmaceutical composition.

A pharmaceutical composition according to the disclosure can be prepared to include a charge-protected oligonucleotide or construct of the disclosure, into a form suitable for administration to a subject using carriers, excipients, and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, anti-oxidants, chelating agents, and inert gases. Other pharmaceutically acceptable carriers include aqueous solutions, non-toxic excipients, including salts, preservatives, buffers and the like, as described, for instance, in Remington's Pharmaceutical Sciences, 15th ed., Easton: Mack Publishing Co., 1405-1412, 1461-1487 (1975), and The National Formulary XIV., 14th ed., Washington: American Pharmaceutical Association (1975), the contents of which are hereby incorporated by reference. The pH and exact concentration of the various components of the pharmaceutical composition are adjusted according to routine skills in the art. See Goodman and Gilman's, The Pharmacological Basis for Therapeutics (7th ed.).

The pharmaceutical compositions according to the disclosure may be administered locally or systemically. By “therapeutically effective dose” is meant the quantity of a fusion polypeptide according to the disclosure necessary to prevent, to cure, or at least partially arrest the symptoms of a disease or disorder (e.g., to inhibit cellular proliferation). Amounts effective for this use will, of course, depend on the severity of the disease and the weight and general state of the subject. Typically, dosages used in vitro may provide useful guidance in the amounts useful for in situ administration of the pharmaceutical composition, and animal models may be used to determine effective dosages for treatment of particular disorders. Various considerations are described, e.g., in Langer, Science, 249: 1527, (1990); Gilman et al. (eds.) (1990), each of which is herein incorporated by reference.

As used herein, “administering a therapeutically effective amount” is intended to include methods of giving or applying a pharmaceutical composition of the disclosure to a subject that allow the composition to perform its intended therapeutic function. The therapeutically effective amounts will vary according to factors, such as the degree of infection in a subject, the age, sex, and weight of the individual. Dosage regima can be adjusted to provide the optimum therapeutic response. For example, several divided doses can be administered daily or the dose can be proportionally reduced as indicated by the exigencies of the therapeutic situation.

The pharmaceutical composition can be administered in a convenient manner, such as by injection (e.g., subcutaneous, intravenous, and the like), oral administration, inhalation, transdermal application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. The composition will typically be sterile and fluid to the extent that easy syringability exists. Typically the composition will be stable under the conditions of manufacture and storage and preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols, such as mannitol, sorbitol, or sodium chloride are used in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

The pharmaceutical composition can be orally administered, for example, with an inert diluent or an assimilable edible carrier. The pharmaceutical composition and other ingredients can also be enclosed in a hard or soft-shell gelatin capsule, compressed into tablets, or incorporated directly into the subject's diet. For oral therapeutic administration, the pharmaceutical composition can be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations can, of course, be varied and can conveniently be between about 5% to about 80% of the weight of the unit.

The tablets, troches, pills, capsules, and the like can also contain the following: a binder, such as gum gragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Of course, any material used in preparing any dosage unit form should be pharmaceutically pure and substantially non-toxic in the amounts employed. In addition, the pharmaceutical composition can be incorporated into sustained-release preparations and formulations.

Thus, a “pharmaceutically acceptable carrier” is intended to include solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the pharmaceutical composition, use thereof in the therapeutic compositions and methods of treatment is contemplated. Supplementary active compounds can also be incorporated into the compositions.

It is especially advantageous to formulate parenteral compositions in dosage unit form for ease of administration and uniformity of dosage. “Dosage unit form” as used herein, refers to physically discrete units suited as unitary dosages for the subject to be treated; each unit containing a predetermined quantity of pharmaceutical composition is calculated to produce the desired therapeutic effect in association with the required pharmaceutical carrier. The specification for the dosage unit forms of the disclosure are related to the characteristics of the pharmaceutical composition and the particular therapeutic effect to be achieve.

The principal pharmaceutical composition is compounded for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

### EXAMPLES

Various examples for synthesis and use are set forth in the attached Figures. In addition, the disclosure provides the following phosphoramidites and structures for neutralizing a charge associated with an anionic oligonucleotides.

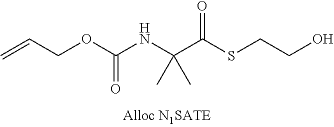

ESI MS for C₁₀H₁₇NO₄S calculated 247.09 observed [M+H]⁺ 247.86

¹H NMR (300 MHz, CDCl₃) δ 1.52 (s, 6H), 2.4 (bs, 1H), 3.08 (m, 2H), 3.74 (m, 2H). 4.56 (d, 2H), 5.2-5.35 (m, 2H), 5.91 (m, 1H).

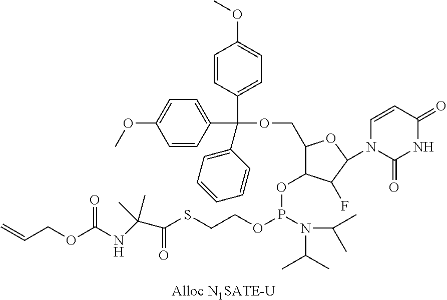

ESI MS for C₄₆H₅₈FN₄O₁₁PS calculated 924.35 observed [M+H]⁺ 925.0 [M+Na]⁺ 947.24

³¹P NMR (121 MHz, CDCl₃) δ 150.09 & 150.14.

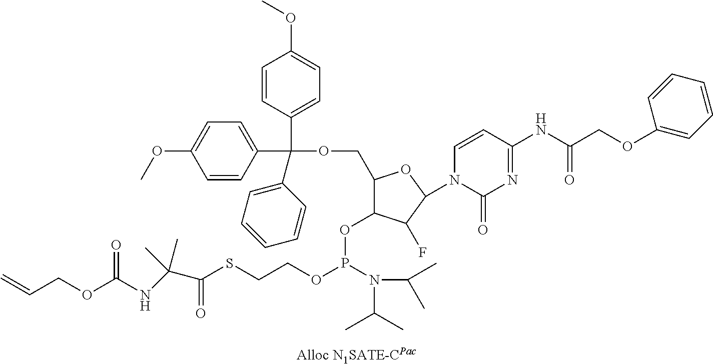

ESI MS for C₅₄H₆₅FN₅O₁₂PS calculated 1057.41 observed [M+H]⁺ 1057.96 [M+Na]⁺ 1080.35

³¹P NMR (121 MHz, CDCl₃) δ 150.08 & 150.12.

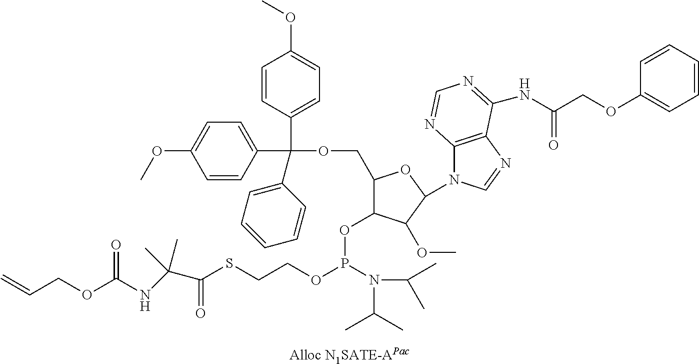

ESI MS for C₅₆H₆₈N₇O₁₂PS calculated 1093.44 observed [M+H]⁺ 1094.25 [M+Na]⁺ 1116.37

³¹P NMR (121 MHz, CDCl₃) δ 150.02 & 150.24.

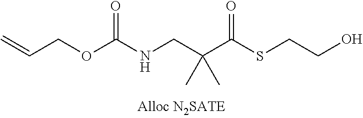

ESI MS for C₁₁H₁₉NO₄S calculated 261.1 observed [M+H]⁺ 262.03

¹H NMR (300 MHz, CDCl₃) δ 1.25 (s, 6H), 2.64 (bs, 1H), 3.07 (m, 2H), 3.35 (m, 2H), 3.74 (m. 2H), 4.52 (m, 2H), 5.18-5.32 (m, 2H), 5.84-5.93 (m, 1H).

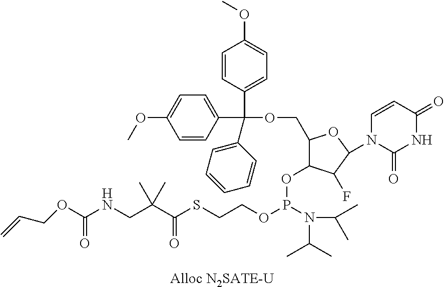

ESI MS for C₄₇H₆₀FN₄O₁₁PS calculated 938.37 observed [M+H]⁺ 939.18 [M+Na]⁺ 961.4

³¹P NMR (121 MHz, CDCl₃) δ 150.37, 150.45 & 150.52.

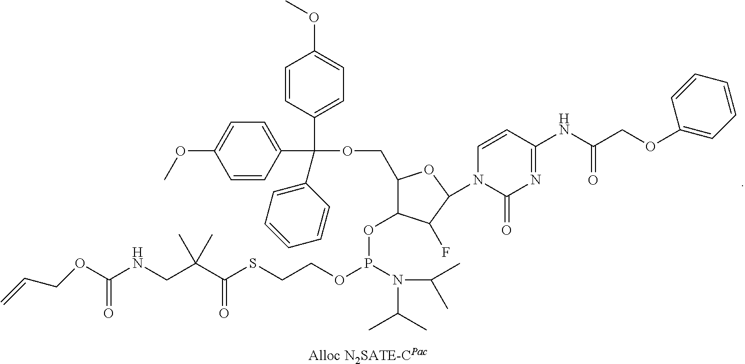

ESI MS for C₅₅H₆₇FN₅O₁₂PS calculated 1071.42 observed [M+H]⁺ 1072.16, [M+Na]⁺ 1094:49

³¹P NMR (121 MHz, CDCl₃) δ 150.29, 150.35, 150.43 & 150.49.

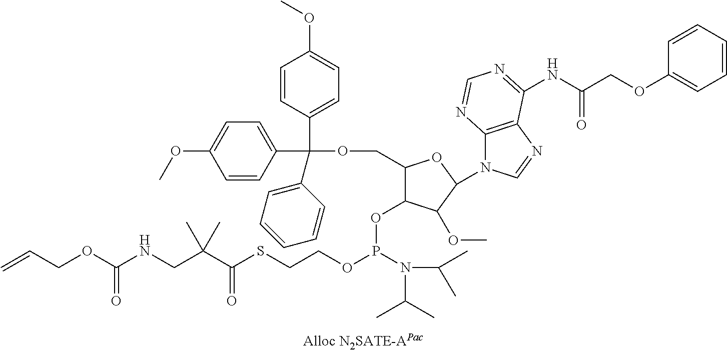

ESI MS for C₅₇H₇₀N₇O₁₂PS calculated 1107.45 observed [M+H]⁺ 1108.36, [M+Na]⁺ 1130.42

³¹P NMR (121 MHz, CDCl₃) δ 150.0 & 150.61.

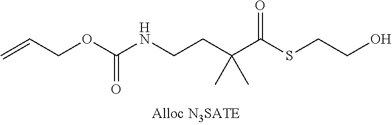

ESI MS for C₁₂H₂₁NO₄S calculated 275.12 observed [M+H]⁺ 276.12

¹H NMR (300 MHz, CDCl₃) δ 1.25 (s, 6H), 1.85 (m, 2H), 3.04 (t, 2H), 3.14 (m, 2H), 3.74 (t, 2H), 4.52 (m, 2H), 5.18-5.31 (m, 2H), 5.88 (m, 1H).

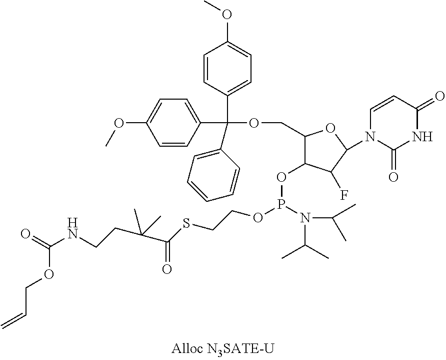

ESI MS for C₄₈H₆₂FN₄O₁₁PS calculated 952.39 observed [M+H]⁺ 953.24, [M+Na]⁺ 975.4.

³¹P NMR (121 MHz, CDCl₃) δ 150.13, 150.20, 150.46 & 150.52.

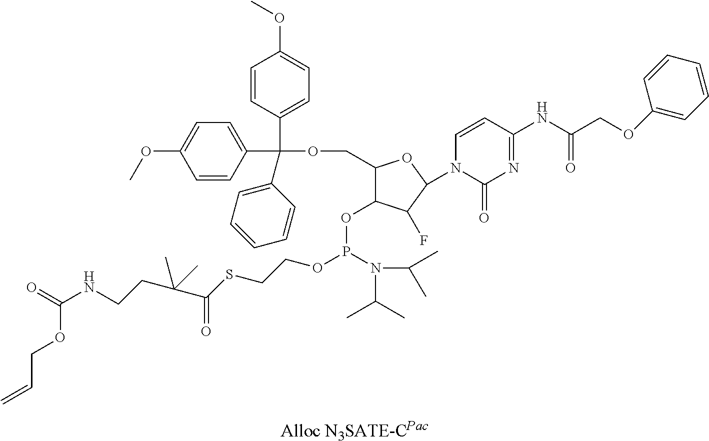

ESI MS for C₅₆H₆₉FN₅O₁₂PS calculated 1085.44 observed [M+H]⁺ 1086.41, [M+H]⁺ 1108.54

³¹P NMR (121 MHz, CDCl₃) δ 150.05, 150.12, 150.19 & 150.23.

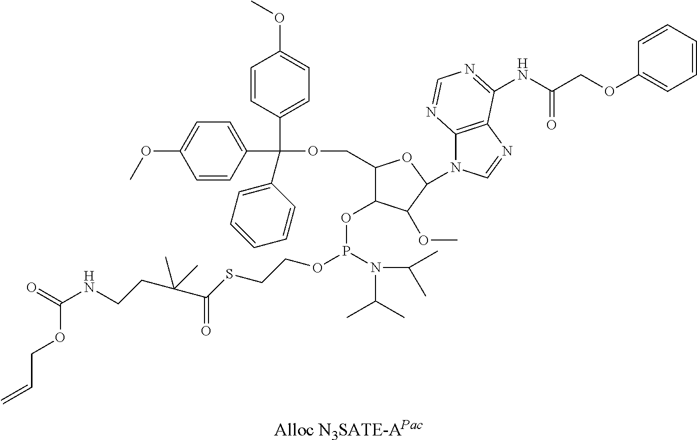

ESI MS for C₅₈H₇₂N₇O₁₂PS calculated 1121.47 observed [M+H]⁺ 1122.22, [M+Na]⁺ 1144.4

³¹P NMR (121 MHz, CDCl₃) δ 150.02 & 150.50.

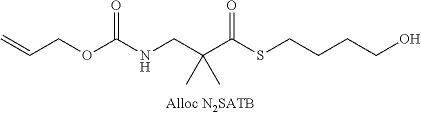

ESI MS for C₁₃H₂₃NO₄S calculated 289.14 observed [M+H]⁺ 290.09 [M+NH₄]⁺ 307.06 [M+Na]⁺ 312.23.

¹H NMR (300 MHz, CDCl₃) δ 1.23 (s, 6H), 1.63 (m, 4H), 2.0 (m, 2H), 2.85-2.89 (m, 2H), 3.31 (m. 2H), 3.64-3.67 (m, 2H), 4.5 (m, 2H), 5.18-5.31 (m, 3H), 5.83-5.92 (m, 1H).

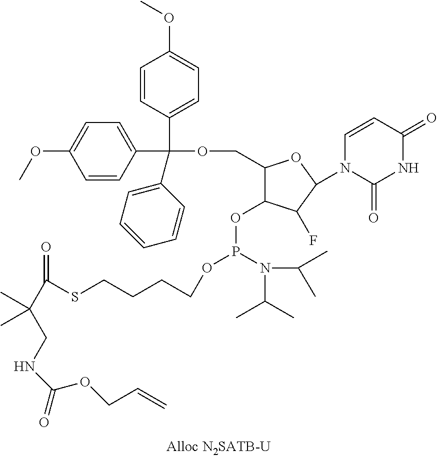

ESI MS for C₄₉H₆₄FN₄O₁₁PS calculated 966.4 observed [M+H]⁺ 967.1 [M+Na]⁺ 989.26 ³¹P NMR (121 MHz, CDCl₃) δ 150.04, 150.12, 150.49 & 150.55.

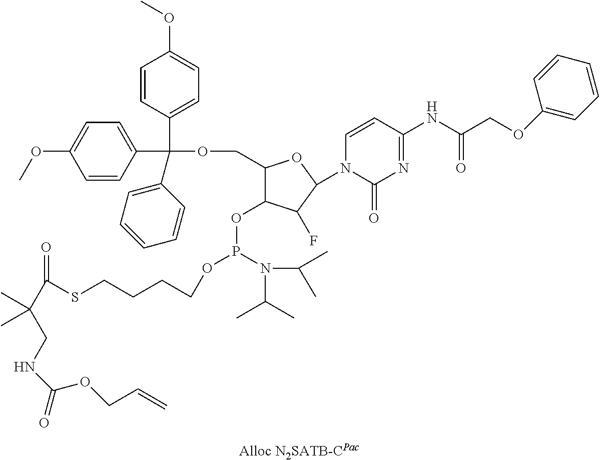

ESI MS for C₅₇H₇₁FN₅O₁₂PS calculated 1099.45 observed [M+H]⁺ 1100.58, [M+Na]⁺ 1122.88

³¹P NMR (121 MHz, CDCl₃) δ 149.98, 150.04, 150.50 & 150.55.

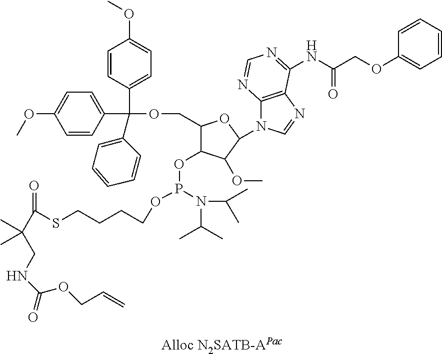

ESI MS for C₅₉H₇₄N₇O₁₂PS calculated 1135.39 observed [M+H]⁺ 1136.5 [M+Na]⁺ 1158.55

³¹P NMR (121 MHz, CDCl₃) δ 149.36 & 150.31.

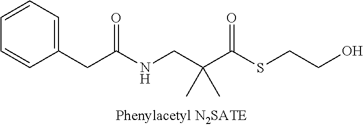

ESI MS for C₁₅H₂₁NO₃S calculated 295.12 observed [M+H]⁺ 296.05

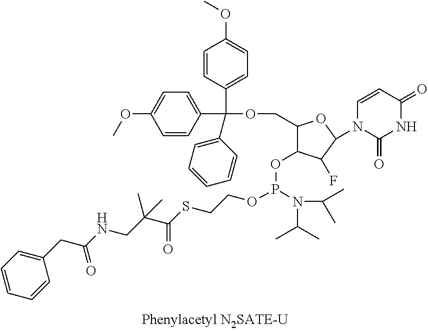

ESI MS for C₅₁H₆₂FN₄O₁₀PS calculated 972.39 observed [M+H]⁺ 973.07

³¹P NMR (121 MHz, CDCl₃) δ 150.51 & 150.58.

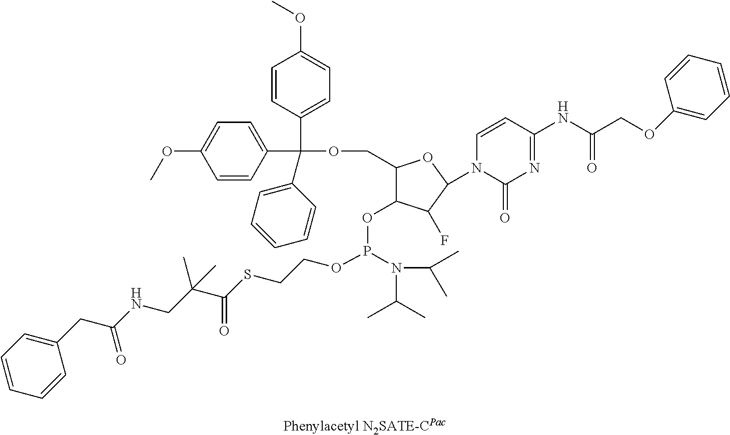

ESI MS for C₅₉H₆₉FN₅O₁₁PS calculated 1105.44 observed [M+H]⁺ 1106.11, [M+Na]⁺ 1128.44.

³¹P NMR (121 MHz, CDCl₃) δ 150.46, 150.51 & 150.58.

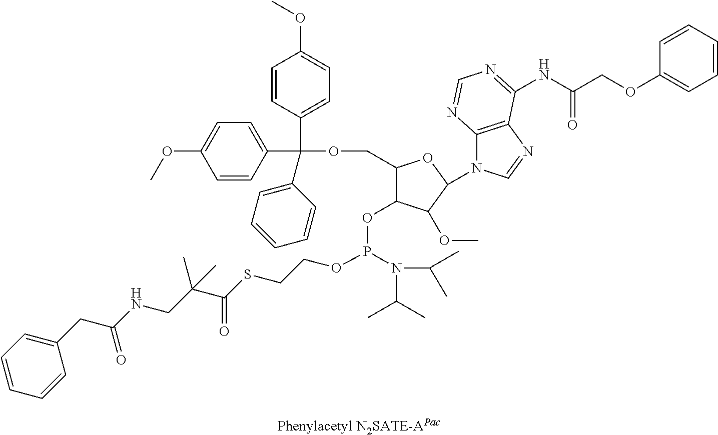

ESI MS for C₆₁H₇₂N₇O₁₁PS calculated 1141.48 observed [M+H]⁺ 1142.14, [M+Na]⁺ 1164.35

³¹P NMR (121 MHz, CDCl₃) δ 150.11 & 150.66.

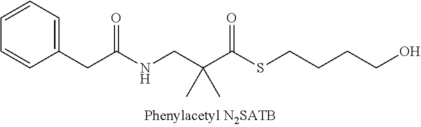

ESI MS for C₁₇H₂₅NO₃S calculated 323.16 observed [M+H]⁺ 324.04

¹H NMR (300 MHz, CDCl₃) δ 1.16 (s, 6H), 1.6 (bs, 4H), 2.16 (s, 2H), 2.8 (t, 2H), 3.35 (m, 2H), 3.6 (t, 2H), 3.66 (m, 2H), 7.23-7.36 (m, 5H).

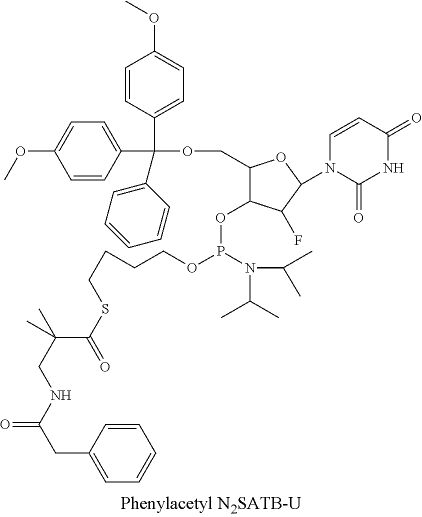

ESI MS for C₅₃H₆₆FN₄O₁₀PS calculated 1000.42 observed [M+H]⁺ 1001.36, [M+Na]⁺ 1023.52

³¹P NMR (121 MHz, CDCl₃) δ 150.19, 150.26, 150.38 & 150.44.

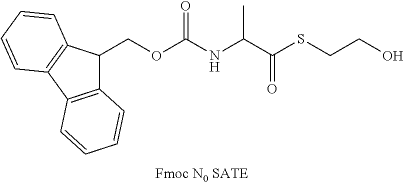

ESI MS for C₂₀H₂₁NO₄S calculated 371.12 observed [M+H]⁺ 371.92, [M+NH₄]⁺ 388.97, [M+Na]⁺ 394.15.

¹H NMR (300 MHz, CDCl₃) δ 1.45 (s, 3H), 2.75-2.92 (m, 2H), 4.25-4.42 (M, 5H), 5.37 (m, 1H). 7.27-7.78 (m, 8H).

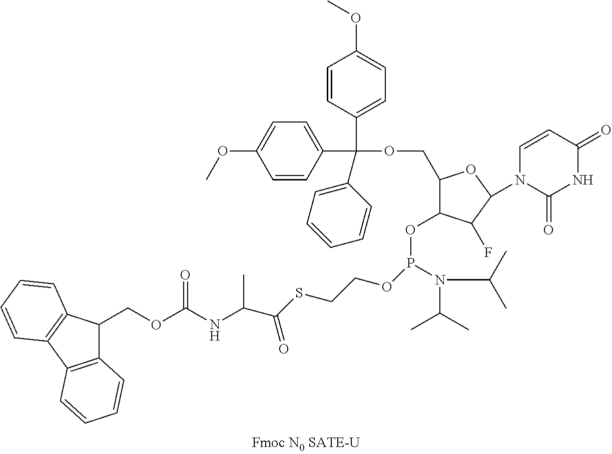

ESI MS for C₅₆H₆₂FN₄O₁₁ PS calculated 1048.39 observed [M+Na]⁺ 1071.47, [M+K]⁺ 1087.4.

³¹P NMR (121 MHz, CDCl₃) δ 169.66, 169.74 & 169.85.

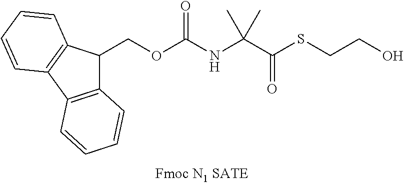

ESI MS for C₂₁H₂₃NO₄S calculated 385.135 observed [M+NH₄]⁺ 403.06, [M+Na]⁺ 408.19

¹H NMR (300 MHz, CDCl₃) δ 1.52 (s, 6H), 2.33 (s, 1H), 3.06 (s. 2H). 3.71 (s, 2H), 4.22 (s, 2H), 4.43 (s, 2H), 5.40 (s, 1H), 7.26-7.78 (m, 8H).

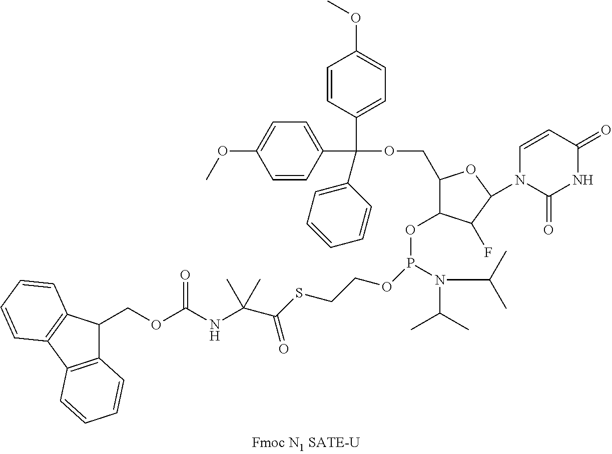

ESI MS for C₅₇H₆₄FN₄O₁₁PS calculated 1062.4 observed [M+H]⁺ 1063.13 [M+Na]⁺ 1085.46

³¹P NMR (121 MHz, CDCl₃) δ 150.23.

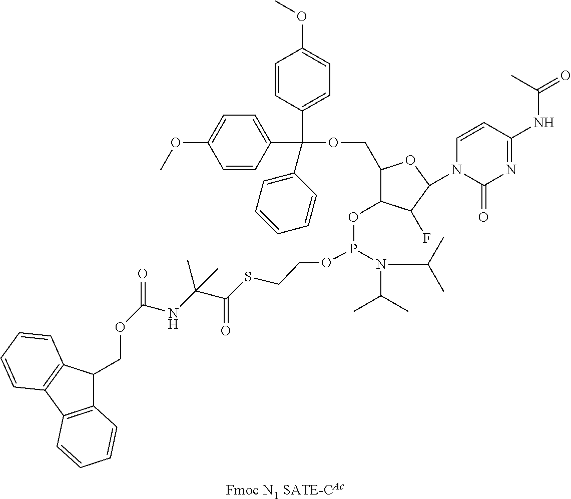

ESI MS for C₅₉H₆₇FN₅O₁₁PS calculated 1103.43 observed [M+H]⁺ 1104.37, [M+Na]⁺ 1126.63

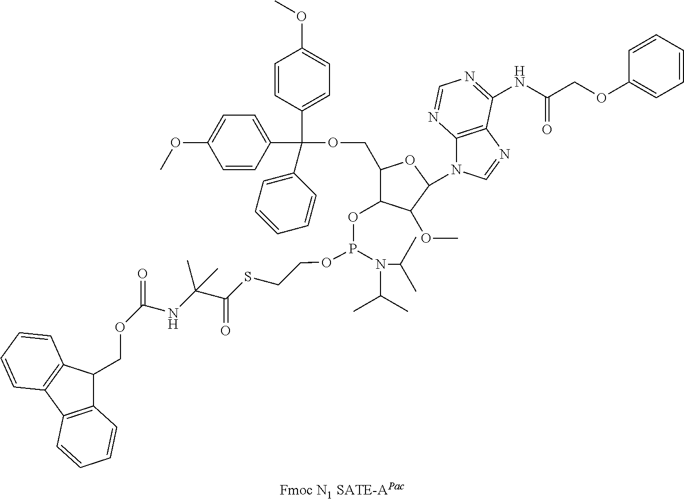

ESI MS for C₆₇H₇₄O₁₂PS calculated 1231.49 observed [M+H]⁺ 1232.31

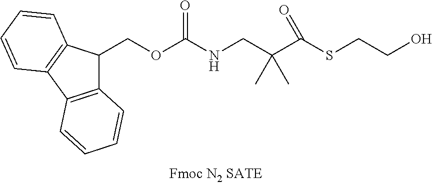

ESI MS for C₂₂H₂₅NO₄S calculated 399.15 observed [M+H]⁺ 399.96, [M+NH₄]⁺ 416.93

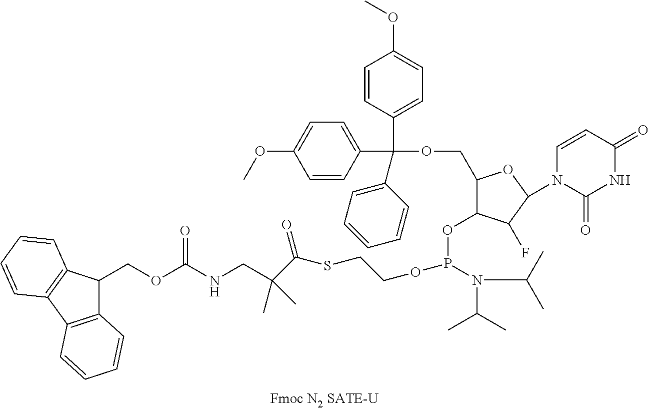

ESI MS for C₅₈H₆₆FN₄O₁₁PS calculated 1076.42 observed [M+H]⁺ 1077.01, [M+Na]⁺ 1099.48

³¹P NMR (121 MHz, CDCl₃) δ 150.4, 150.47, 150.6 & 150.67.

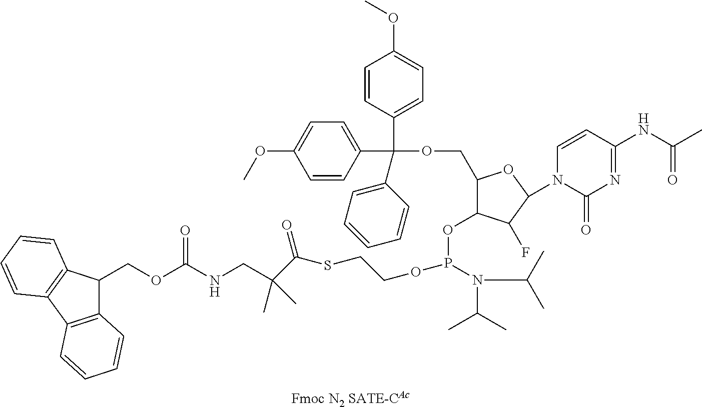

ESI MS for C₆₀H₆₉FN₅O₁₁PS calculated 1117.44 observed [M+Na]⁺ 1140.59

³¹P NMR (121 MHz, CDCl₃) δ 150.27, 150.32, 150.61 & 150.67.

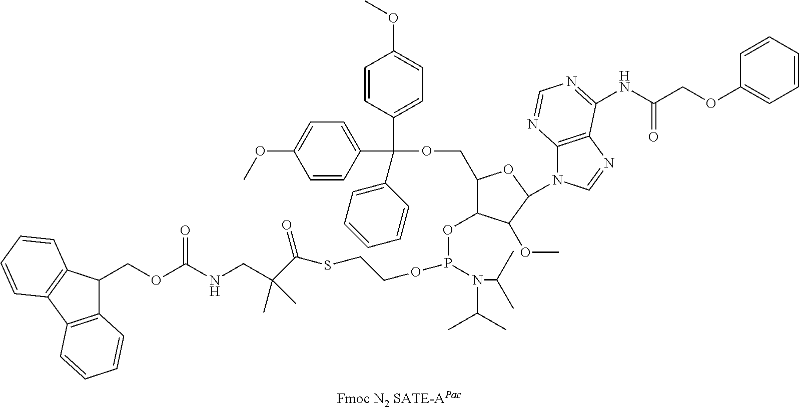

ESI MS for C₆₈H₇₆N₇O₁₂PS calculated 1245.5 observed [M+H]⁺ 1268.65

³¹P NMR (121 MHz, CDCl₃) δ 150.04 & 150.69.

A number of embodiments of the disclosure have been described. Nevertheless, it will be understood that various modifications may be made without departing from the spirit and scope of the disclosure. Accordingly, other embodiments are within the scope of the following claims. cm 1. A nucleotide compound comprising an amino alkyl S-acyl thio alkyl (N-SATE) moiety conjugated to the phosphate group of the nucleotide.

2. The nucleotide of claim 1, wherein the N-SATE moiety comprises the general structure: 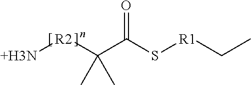
wherein R₁ may or may not be present, when R₁ is present, R₁ is selected from the group consisting of alkyl, substituted alkyl, alkoxy, substituted alkoxy, cycloalkyl, substituted cycloalkyl, alkenyl, substituted alkenyl, alkynyl, substituted alkynyl, aryl, substituted aryl, heterocyclic, or substituted heterocyclic;
wherein R₂ may or may not be present, when R₂ is present, R₂ is selected from the group consisting of a 1 to 7 atom alkyl, substituted alkyl, alkoxy, substituted alkoxy, cycloalkyl, substituted cycloalkyl, alkenyl, substituted alkenyl, alkynyl, substituted alkynyl, aryl, substituted aryl, heterocyclic, or substituted heterocyclic.

3. The nucleotide of claim 2, wherein the N-SATE moiety is selected from the group consisting of:

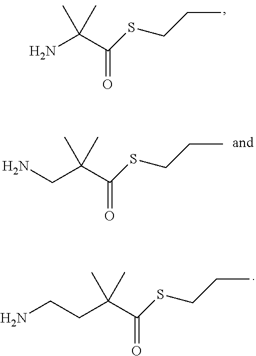

4. The nucleotide compound of claim 2, wherein the N-SATE moiety is conjugated to the phosphate group of any of the nucleic acid bases A, G, C, T or U.

5. The nucleotide of claim 1, wherein when the nucleotide is linked through a phosphate bond to another nucleotide a linked backbone comprises the general structure: 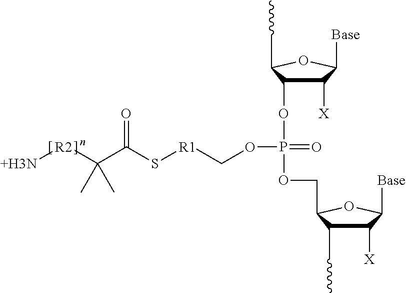
wherein R₁ may or may not be present, when R₁ is present, R₁ is selected from the group consisting of alkyl, substituted alkyl, alkoxy, substituted alkoxy, cycloalkyl, substituted cycloalkyl, alkenyl, substituted alkenyl, alkynyl, substituted alkynyl, aryl, substituted aryl, heterocyclic, or substituted heterocyclic;
wherein R₂ may or may not be present, when R₂ is present, R₂ is selected from the group consisting of a 1 to 7 atom alkyl, substituted alkyl, alkoxy, substituted alkoxy, cycloalkyl, substituted cycloalkyl, alkenyl, substituted alkenyl, alkynyl, substituted alkynyl, aryl, substituted aryl, heterocyclic, or substituted heterocyclic.

6. The nucleotide of claim 5, wherein the N-SATE moiety is selected from the group consisting of:

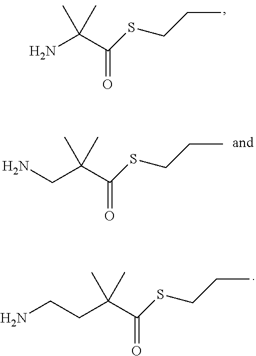

7. An oligonucleotide or polynucleotide comprising a nucleotide having an N-SATE moiety of claim 1.

8. The oligonucleotide or polynucleotide of claim 7, wherein the oligonucleotide or polynucleotide comprises a neutral or a more cationic charge when compared to the same oligonucleotide or polynucleotide lacking an N-SATE moiety.

9. An oligonucleotide or polynucleotide comprising an amino alkyl S-acyl thio alkyl (N-SATE) moiety that reduces the net anionic charge of the oligonucleotide or polynucleotide backbone.

10. The oligonucleotide or polynucleotide of claim 9, wherein the oligonucleotide or polynucleotide comprise an siRNA molecule.

11. The oligonucleotide or polynucleotide of claim 9, wherein the oligonucleotide comprises a plurality modified nucleotides having an N-SATE moiety.

12. The oligonucleotide or polynucleotide of claim 11, wherein the oligonucleotide or polynucleotide comprises a plurality of adjacent nucleotides having an N-SATE moiety.

13. The oligonucleotide or polynucleotide of claim 11, wherein the oligonucleotide or polynucleotide comprises a plurality of nucleotides having an N-SATE moiety separated from one another by 1 or more nucleotide bases.

14. The oligonucleotide or polynucleotide of claim 9, further comprising at least one protein transduction domain (PTD) comprising a membrane transport function conjugated or operably linked to the oligonucleotide or polynucleotide domain.

15. The oligonucleotide of claim 14 comprising a plurality of protein transduction domains.

16. A pharmaceutical composition comprising the oligonucleotide or polynucleotide of claim 9.

17. A method of delivering an oligonucleotide or polynucleotide to a cell in vitro or in vivo comprising contacting the cell with the pharmaceutical composition of claim 16.

18. A method of making a charge neutralized or cationically charged oligonucleotide of claim 9 comprising chemically synthesizing the oligonucleotide in a synthesizer using a phosphoramidite having the general structure

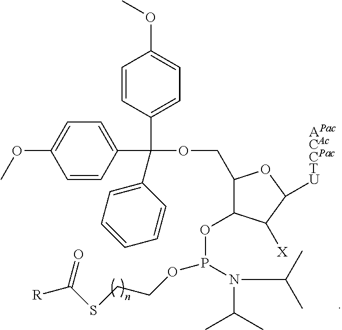

19. The method of claim 18, wherein the synthesizer is an RNA synthesizer.

20. A method of delivering an oligonucleotide or polynucleotide to a cell in vitro or in vivo comprising contacting the cell with the oligonucleotide or polynucleotide of claim 9.


SEQUENCE LISTING (25 sequences)

SEQ ID NO: 1 - protein, 8 aa, Artificial Sequence
  Protein Transduction Domain Consensus Sequence
XXXXXXXX

SEQ ID NO: 2 - protein, 8 aa, Artificial Sequence
  Protein Transduction Domain Consensus Sequence
XXXXXXXX

SEQ ID NO: 3 - protein, 7 aa, Artificial Sequence
  Protein Transduction Domain
KKRPKPG

SEQ ID NO: 4 - protein, 11 aa, Artificial Sequence
  Protein Transduction Domain Consensus Sequence
XXRXXXXXXXX

SEQ ID NO: 5 - protein, 5 aa, Artificial Sequence
  Protein Transduction Domain Consensus Sequence
KXRXX

SEQ ID NO: 6 - protein, 9 aa, Artificial Sequence
  Protein Transduction Domain
RKKRRQRRR

SEQ ID NO: 7 - protein, 86 aa, Human immunodeficiency virus
MEPVDPRLEPWKHPGSQPKTACTNCYCKKCCFHCQVCFITKALGISYGRKKRRQRRRPPQGSQTHQVSLSKQPTSQSRGDPTGPKE

SEQ ID NO: 8 - protein, 5 aa, Artificial Sequence
  Peptide Linker
GGGGS

SEQ ID NO: 9 - protein, 12 aa, Artificial Sequence
  Peptide Linker
GKSSGSGSESKS

SEQ ID NO: 10 - protein, 14 aa, Artificial Sequence
  Peptide Linker
GSTSGSGKSSEGKG

SEQ ID NO: 11 - protein, 18 aa, Artificial Sequence
  Peptide Linker
GSTSGSGKSSEGSGSTKG

SEQ ID NO: 12 - protein, 18 aa, Artificial Sequence
  Peptide Linker
GSTSGSGKPGSGEGSTKG

SEQ ID NO: 13 - protein, 14 aa, Artificial Sequence
  Peptide Linker
EGKSSGSGSESKEF

SEQ ID NO: 14 - protein, 11 aa, Artificial Sequence
  PTD sequence
AGRKKRRQRRR

SEQ ID NO: 15 - protein, 11 aa, Artificial Sequence
  PTD sequence
YARKARRQARR

SEQ ID NO: 16 - protein, 11 aa, Artificial Sequence
  PTD sequence
YARAAARQARA

SEQ ID NO: 17 - protein, 11 aa, Artificial Sequence
  PTD sequence
YARAARRAARR

SEQ ID NO: 18 - protein, 11 aa, Artificial Sequence
  PTD sequence
YARAARRAARA

SEQ ID NO: 19 - protein, 11 aa, Artificial Sequence
  PTD sequence
YARRRRRRRRR

SEQ ID NO: 20 - protein, 11 aa, Artificial Sequence
  PTD sequence
YAAARRRRRRR

SEQ ID NO: 21 - DNA, 20 bp, Artificial Sequence
  Synthesized oligonucleotide
tttttttttttttttuuutt

SEQ ID NO: 22 - DNA, 21 bp, Artificial Sequence
  Synthesized oligonucleotide
tuggugauggacucguggguc

SEQ ID NO: 23 - DNA, 42 bp, Artificial Sequence
  Synthesized oligonucleotide
ccacuaccugagcacccagtttuggugauggacucguggguc

SEQ ID NO: 24 - DNA, 21 bp, Artificial Sequence
  Synthesized oligonucleotide
cugggugcucagguaguggtt

SEQ ID NO: 25 - DNA, 21 bp, Artificial Sequence
  Synthesized oligonucleotide
ccacuaccugagcacccagtt